(19) United States
(12) Patent Application Publication
(10) Pub. No.: US 2026/0207504 A1
(43) Pub. Date: Jul. 23, 2026

(54) STABILIZATION OF LIPID NANOPARTICLE FORMULATIONS

(71) Applicant: Eli Lilly and Company, Indianapolis, IN (US)

(72) Inventors: Daniel Estabrook, Indianapolis, IN (US); Zhao Yu, Indianapolis, IN (US); Lihua Huang, Indianapolis, IN (US); Tingting Wang, Indianapolis, IN (US)

(73) Assignee: Eli Lilly and Company, Indianapolis, IN (US)

(21) Appl. No.: 19/141,578

(22) PCT Filed: Dec. 15, 2023
(86) PCT No.: PCT/US2023/084459

§ 371 Date: Jun. 20, 2025

Related U.S. Application Data
(60) Provisional application No. 63/435,024, filed on Dec. 23, 2022.

(51) Int. Cl. A61K 9/1272; A61K 31/7105; A61K 47/02; A61K 47/12; A61K 47/18; A61K 47/22

(52) U.S. Cl. CPC A61K 9/1272; A61K 31/7105; A61K 47/02; A61K 47/12; A61K 47/186; A61K 47/22

(57) ABSTRACT

Aspects of the disclosure relate to compositions and methods for improving the stability of lipid nanoparticles (LNPs). In some embodiments, the LNPs comprise one or more active pharmaceutical ingredients (API) encapsulated therein. The disclosure is based, in part, on compositions that directly or indirectly reduce the degradation (e.g., oxidation, hydrolysis, etc.) of one or more lipids components of the lipid nanoparticle. In some embodiments, the compositions comprise a histidine buffer. The disclosure also provides methods for storing compositions contemplated herein as well as methods for improving the stability of the API.

### RELATED APPLICATIONS

This application is a national stage filing under 35 U.S.C. § 371 of international PCT application PCT/US2023/084459, filed Dec. 15, 2023, which claims priority under 35 U.S.C. § 119(e) to U.S. provisional patent application, U.S. Ser. No. 63/435,024, filed Dec. 23, 2022, the entire contents of each of which are incorporated by reference herein.

### BACKGROUND

Lipid nanoparticle (LNP) formulations are useful drug delivery systems for encapsulating various active pharmaceutical ingredients (APIs), for example, RNAs. LNP formulations commonly include aqueous buffer solutions, for example, phosphate buffer. However, current LNP formulations are susceptible to degradation and aggregate formation during storage at room temperature, thus improvements are needed.

### SUMMARY

Aspects of the disclosure relate to compositions and methods for improving the stability of lipid nanoparticles (LNPs) and one or more active pharmaceutical ingredients (API) encapsulated therein. The disclosure is based, in part, on compositions that directly or indirectly reduce the degradation (e.g., oxidation, hydrolysis, etc.) of one or more lipids components of the lipid nanoparticle. In some embodiments, the compositions comprise a histidine buffer. In some embodiments, the histidine buffer inhibits oxidation and/or hydrolysis of one or more lipids of the LNP and/or improves the stability of an API encapsulated within the LNP. The disclosure also provides methods for storing compositions contemplated herein. Accordingly, in some aspects, the disclosure provides a pharmaceutical composition comprising a lipid nanoparticle (LNP) comprising one or more ionizable lipids, and a histidine buffer having a concentration ranging from 5 mM to 30 mM histidine and a pH ranging from about 5.0 to about 7.5.

In some embodiments, the concentration of histidine buffer ranges between 10 mM and 20 mM histidine. In some embodiments, the concentration of histidine buffer is at least 10 mM. In some embodiments, the concentration of histidine buffer is 20 mM.

In some embodiments, the pH of the histidine buffer is about pH 6.0.

In some embodiments, the histidine buffer further comprises one or more salts. In some embodiments, the one or more salts comprises NaCl. In some embodiments, the histidine buffer comprises one or more non-ionic excipients, for example sucrose.

In some embodiments, the one or more ionizable lipids comprises an unsaturated tail, further optionally wherein the one or more ionizable lipids comprises DLin-MC3-DMA (MC3) and/or Dioleoyl-3-trimethylammonium propane (DOTAP). In some embodiments, the one or more ionizable lipid consists of MC3 or DOTAP.

In some embodiments, the LNP comprises one or more nucleic acids. In some embodiments, the one or more nucleic acids comprises RNA. In some embodiments, the RNA is mRNA, siRNA, dsRNA, or miRNA. In some embodiments, the RNA is siRNA.

In some embodiments, the composition has not been refrigerated or frozen (e.g., after addition of the histidine buffer). In some embodiments, the composition is stored at a temperature above 4° C. In some embodiments, the temperature ranges from about 5° C. to about 30° C. In some embodiments, the composition is frozen (e.g., before or after the addition of histidine buffer), and subsequently thawed and stored at a temperature that ranges from about 5° C. to about 30° C.

In some aspects, the disclosure provides a container containing a pharmaceutical composition as described herein. In some embodiments, the container is a cartridge, prefilled syringe, or glass vial. In some embodiments, the container is a prefilled syringe or a glass vial. In some embodiments, the container is a polymer vial.

In some aspects, the disclosure provides a method for improving chemical stability of a lipid nanoparticle (LNP) pharmaceutical composition, the method comprising obtaining a non-histidine-buffered LNP pharmaceutical composition comprising a non-histidine buffer; and performing a buffer exchange procedure to replace the non-histidine buffer with a histidine buffer having a pH between 5.8 and 7.5 to obtain a histidine-buffered LNP pharmaceutical composition.

In some embodiments, the histidine-buffered LNP pharmaceutical composition comprises one or more ionizable lipids (e.g., one or more ionizable lipids having an unsaturated fatty acid tail). In some embodiments, the ionizable lipids comprise MC3 and/or DOTAP. In some embodiments, the ionizable lipids consist of MC3 or DOTAP.

In some embodiments, the non-histidine buffer comprises a citrate buffer. In some embodiments, the citrate buffer has a pH ranging from about 3.5 to about 5.5. In some embodiments, the citrate buffer has a pH of 5.0.

In some embodiments, the histidine-buffered LNP pharmaceutical composition comprises one or more nucleic acids. In some embodiments, the one or more nucleic acids comprises RNA. In some embodiments, the RNA is mRNA, siRNA, dsRNA, or miRNA. In some embodiments, the RNA is siRNA.

In some embodiments, the histidine buffer has a pH of 6.0. In some embodiments, the histidine buffer has a concentration of histidine ranging from about 5 mM to about 30 mM. In some embodiments, the histidine buffer has a concentration of histidine ranging from about 10 mM and 20 mM. In some embodiments, the concentration of histidine is at least 10 mM. In some embodiments, the concentration of histidine is 20 mM.

In some embodiments, the buffer exchange procedure comprises contacting the non-histidine-buffered LNP pharmaceutical composition to a de-salting column. In some embodiments, the buffer exchange procedure comprises contacting the non-histidine-buffered LNP pharmaceutical composition to a dialysis tube or performing tangential flow filtration. In some embodiments, the buffer exchange reaction comprises contacting the non-histidine-buffered LNP pharmaceutical composition to tangential flow filtration.

In some embodiments, the buffer exchange procedure comprises collecting the histidine-buffered LNP pharmaceutical composition in a container (e.g., a vial or syringe). In some embodiments, the container is a cartridge, prefilled syringe, or glass vial. In some embodiments, the vial is a glass vial. In some embodiments, the vial is a polymer vial.

In some embodiments, the method further comprises storing the histidine-buffered LNP pharmaceutical composition at a temperature above 4° C. In some embodiments, the temperature ranges from about 5° C. to about 30° C.

In some embodiments, the histidine-buffered LNP pharmaceutical composition comprises fewer hydrolyzed lipids relative to a non-histidine-buffered LNP pharmaceutical composition stored in a phosphate buffer.

In some embodiments, the histidine-buffered LNP pharmaceutical composition comprises fewer oxidized lipids relative to a non-histidine-buffered LNP pharmaceutical composition stored in a phosphate buffer.

In some embodiments, the histidine-buffered LNP pharmaceutical composition comprises LNPs having increased colloidal stability relative to non-histidine-buffered LNPs stored in a pharmaceutical composition comprising phosphate buffer.

In some aspects, the disclosure provides a method for preparing a lipid nanoparticle (LNP) pharmaceutical composition, the method comprising preparing a first lipid composition comprising one or more lipids; preparing a second lipid composition comprising a siRNA and an ionizable lipid, and mixing the first lipid composition and the second lipid composition, wherein the first lipid composition and second lipid composition are prepared using a histidine buffer having a pH between 5.8 and 6.5 and a histidine concentration of between 5 mM to 30 mM.

In some aspects, the disclosure provides a method for preparing a lipid nanoparticle (LNP) pharmaceutical composition, the method comprising obtaining a lipid composition comprising a siRNA and an ionizable lipid, and; mixing the lipid composition with a histidine buffer having a pH between 5.8 and 6.5 and a histidine concentration of between 5 mM to 30 mM.

### BRIEF DESCRIPTION OF DRAWINGS

FIG. 1 shows representative structural features of two ionizable lipids, MC3 and DOTAP, used to manufacture lipid nanoparticles (LNPs).

FIGS. 2A and 2B show the percentage of DOTAP (FIG. 2A) and MC3 (FIG. 2B) lipids, respectively, that are intact over a period of four weeks following storage in either glass (BT5933) or cyclic olefin polymer (COP) vials at different temperatures. Briefly, DOTAP or MC3 was dissolved in ethanol at 4 mg/mL concentration. Then, the solution was diluted in 1×PBS buffer and filled into either glass vials or COP vials.

FIGS. 3A-3C show representative data indicating percentage hydrolysis (FIG. 3A) and percentage oxidation (FIG. 3B) of DOTAP lipids, and percentage hydrolysis (FIG. 3C) of MC3 lipids over a period of four weeks following storage in either glass (BT5933) or cyclic olefin polymer (COP) vials at different temperatures.

FIGS. 4A and 4B show representative data indicating storage of DOTAP lipids in histidine buffers improves the percentage of intact DOTAP lipids after storage across a range of temperatures over four weeks (FIG. 4A) and that histidine buffers reduce hydrolysis of DOTAP lipids during storage across a range of temperatures over four weeks (FIG. 4B). Histidine buffer has more significant impact on the chemical stability of DOTAP than container/closure systems

FIGS. 5A-5C show representative data indicating storage of MC3 lipids in histidine buffers improves the percentage of intact MC3 lipids after storage across a range of temperatures over four weeks (FIG. 5A) and that histidine buffers reduce hydrolysis (FIG. 5B) and oxidation (FIG. 5C) of MC3 lipids during storage across a range of temperatures over four weeks. Histidine buffer has more significant impact on the chemical stability of MC3 than container/closure systems.

FIGS. 6A and 6B show representative data indicating storage of empty MC3-LNPs in histidine buffers stabilizes LNP particle size when stored at 5° C. (FIG. 6A) and room temperature (RT) over a 4-week period (FIG. 6B). Histidine buffer is used in 5933 His samples, while 1× PBS buffer is used in other samples. BT5933 is type 1 glass vial with coefficient of expansion (COE) of 51. BT5974 vial is type 1 glass vial with COE of 33.

FIGS. 7A and 7B show representative data indicating storage of empty DOTAP-LNPs in histidine does not substantially alter LNP size compared to PBS buffer following storage at either 5° C. or RT for up to 4 weeks. Histidine buffer is used in 5933 His samples, while 1× PBS buffer is used in other samples.

FIGS. 8A and 8B show representative data indicating storage of empty MC3-LNPs in histidine buffers stabilizes the percentage of intact MC3 across a range of temperatures over four weeks (FIG. 8A) and decreases oxidation of MC3 in the LNPs (FIG. 8B).

FIGS. 9A and 9B show representative data indicating storage of empty DOTAP-LNPs in histidine buffers stabilizes the percentage of intact DOTAP across a range of temperatures over four weeks (FIG. 9A) and decreases hydrolysis of DOTAP in the DOTAP-LNPs (FIG. 9B) across a range of temperatures over four weeks.

FIGS. 10A and 10B show representative data indicating stabilization of siRNA-loaded MC3-LNP particle diameter (FIG. 10A) and polydispersity index (FIG. 10B) following formulation in histidine buffer compared to formulation in PBS buffer across a range of temperatures over four weeks. All samples were stored in glass vials.

FIGS. 11A and 11B show representative data indicating histidine buffer storage stabilizes MC3-LNP encapsulation of siRNA as a function of time compared to PBS buffer across a range of temperatures over four weeks (FIG. 11A) and reduces subvisible particle concentration as measured by micro-flow imaging (MFI) after four weeks across a range of temperatures (FIG. 11B).

FIGS. 12A and 12B show representative data indicating the relative percentage of intact MC3 in MC3-LNPs encapsulating siRNAs formulated in PBS (FIG. 12A) or histidine buffers (FIG. 12B) across a range of temperatures over four weeks.

FIGS. 13A and 13B show representative data indicating the stability of siRNA sense and antisense strands encapsulated within MC3-LNPs formulated in either PBS (FIG. 13A) or histidine buffers (FIG. 13B) across a range of temperatures over four weeks.

FIG. 14 shows representative data indicating the dose-dependent function of siRNA-loaded into MC3-LNPs formulated in either PBS or histidine buffers and stored across a range of temperatures over four weeks.

FIG. 15 shows histidine buffered siRNA-LNPs remained significantly more stable than phosphate buffered compositions after 3 months storage at RT.

FIG. 16A shows that storage in histidine buffer results in formation of fewer siRNA-lipid adducts during storage at a variety of temperatures relative to phosphate buffered compositions.

FIG. 16B shows representative data indicating that histidine buffer storage inhibits oxidation of phosphorothioate linkages (PS) to phosphodiester (PO) linkages in chemically-modified siRNAs.

FIGS. 17A-17D show representative data for measurement of mRNA-LNP colloidal and payload stability. FIG. 17A shows mRNA-LNP size at 25° C. FIG. 17B shows mRNA-LNP polydispersity index (PDI) at 25° C. FIG. 17C shows mRNA-LNP encapsulation efficiency (EE) at 25° C. FIG. 17D shows mRNA-LNP RNA content at 25° C. RNA content was observed to be reduced in PBS stored compositions.

FIG. 18 shows representative data indicating that MC3-stabilized mRNA-LNPs experience a similar level of ionizable lipid degradation as siRNA-LNPs, and that storage in histidine buffer prevents lipid degradation.

FIG. 19 shows representative CryoEM images.

### DETAILED DESCRIPTION

Aspects of the disclosure relate to compositions and methods for improving the stability of lipid nanoparticles and one or more active pharmaceutical ingredients (API) encapsulated therein. The disclosure is based, in part, on compositions comprising components (e.g., histidine buffers at certain pH ranges, etc.) that directly or indirectly reduce the degradation (e.g., oxidation, hydrolysis, etc.) of one or more lipids components of the LNP. The disclosure also provides methods for storing compositions contemplated herein as well as methods for improving the stability of the API.

### Lipid Nanoparticles

Aspects of the disclosure relate to compositions comprising lipid nanoparticles (LNPs). As used herein, the term “LNP” refers to any particle comprising at least one lipid component having an average mean diameter of less than 1000 nanometers. Exemplary LNPs include, but are not limited to, micelles, liposomes, lipoplexes, and solid lipid nanoparticles, or derivatives thereof. LNPs may have any morphology and structure known in the art. For example, in some embodiments, LNPs are nanospheres, nanorods, nanochains, nanostars, nanoflowers, nanoreefs, nanowhiskers, nanofibers, and nanoboxes. In some embodiments, other morphologies and structures are also possible.

In some embodiments, an LNP comprises a micelle. A micelle may be a micelle or a reverse-micelle. In some embodiments, micelles are aggregates of amphipathic lipids dispersed in a liquid forming a colloidal suspension. In some embodiments, amphipathic lipids comprise a hydrophilic head group and a hydrophobic tail group that, when dispersed in a water, spontaneously assembles into structures that expose the head groups to the water phase and bury the hydrophobic tails group into the core of the structure. In some embodiments, the lipids are dispersed into an oil phase and spontaneously assemble into structures that expose the hydrophobic tail groups and bury the hydrophilic head groups (e.g., the lipids form a reverse micelle). In some embodiments, combinations of structures are also possible. For example, in some embodiments, lipids may be added to emulsions, for example, a water-in-oil emulsion, an oil-in-water emulsion, or the like. In some embodiments, lipids added to an oil-in-water emulsion (e.g., water is the continuous phase and oil is the dispersed phase) spontaneously assemble at the water-oil interface with the hydrophilic heads pointed toward the water phase and the hydrophilic tails oriented toward the oil phase. In this configuration, the lipids form a monolayer around the oil droplet transforming it from a hydrophobic surface into a hydrophilic surface, thus stabilizing the oil droplets in the water phase (e.g., preventing the oil droplets from coalescing). In some embodiments, lipids added to water-in-oil emulsion (e.g., oil is the continuous phase and water is the dispersed phase) from a monolayer around the water droplets dispersed in an oil phase. In some embodiments, the lipid monolayer may comprise one or more targeting molecules (e.g., antibodies or fragments thereof, cell targeting peptides, etc.), drugs, or other agents (e.g., polyethylene glycol). Combinations of emulsions are also possible in some embodiments. For example, in some embodiments, water-in-oil-in-water emulsion are contemplated herein. Such a configuration would produce stabilized water droplets encapsulated within a larger oil droplet. Other combinations are also possible in some embodiments. For example, in some embodiments, oil-in-water-in-oil emulsions may be used to create the LNPs disclosed herein.

In some embodiments, the LNP comprises a liposome. In some embodiments, a liposome is an artificial vesicle having at least one lipid bilayer. In some embodiments, the liposome is a multilamellar vesicle (MLV). In some embodiments, the liposome is a large unilamellar vesicle (LUV). In some embodiments, the liposome is a small unilamellar vesicle (SUV). In some embodiments, MLVs are large “onion-like” structures comprising several lamellar phase lipid bilayers (e.g., mean diameters greater than 1000 nanometers). In some embodiments, LUVs comprise large unilamellar vesicles (e.g., average dimeters of between 100 to 200 nanometers). In some embodiments, SUVs comprise small unilamellar vesicles (e.g., mean diameter of between 15 to 30 nanometers). In some embodiments, the lipid bilayer may comprise one or more targeting molecules (e.g., antibodies or fragments thereof, cell targeting peptides, etc.), drugs, or other agents (e.g., polyethylene glycol).

In some embodiments, the LNP comprises a lipoplex. As used herein, the term “lipoplex” is refers to a complex formed between a charged liposome (e.g., a cationic or anionic lipid) and at least one oppositely charged component (e.g., a nucleic acid, such as a siRNA). As used herein, the at least one oppositely charged component may be a small molecule drug, a polynucleotide (e.g., DNA, RNA, siRNA, miRNA, etc.), a peptide, a polypeptide, a protein (e.g., an antibody), a polymer, or a polysaccharide comprising at least one cationic (e.g., —NH3+) and/or anionic (e.g., COO—) group. Without wishing to be bound by theory, it is believed that the complex is formed due to the electrostatic interaction between the opposing charge groups between the charged lipid in the liposome bilayer and the oppositely charged component.

In some embodiments, any one of the LNPs disclosed herein comprise solid lipid nanoparticles. In some embodiments, solid lipid nanoparticles comprise a solid lipid core matrix capable of solubilizing lipophilic molecules (e.g., oils, lipids, charged lipid-complexes, DNA, RNA, etc.). In some embodiments, the solid lipid core is stabilized by a lipid monolayer (e.g., similar to micelles). In some embodiments, the lipid monolayer may comprise one or more targeting molecules (e.g., antibodies or fragments thereof, cell targeting peptides, etc.), drugs, or other agents (e.g., polyethylene glycol).

In some embodiments, the LNPs of the present disclosure have an average mean diameter of greater than or equal to 1 nanometer, greater than or equal to 5 nanometers, greater than or equal to 10 nanometers, greater than or equal to 50 nanometers, greater than or equal to 100 nanometers, greater than or equal to 200 nanometers, greater than or equal to 300 nanometers, greater than or equal to 400 nanometers, greater than or equal to 500 nanometers, greater than or equal to 600 nanometers, greater than or equal to 700 nanometers, greater than or equal to 800 nanometers, greater than or equal to 900 nanometers, greater than or equal to 1000 nanometers. In some embodiments, the LNPs have an average mean diameter of less than or equal to 1000 nanometers, less than or equal to 900 nanometers, less than or equal to 800 nanometers, less than or equal to 700 nanometers, less than or equal to 600 nanometers, less than or equal to 500 nanometers, less than or equal to 400 nanometers, less than or equal to 300 nanometers, less than or equal to 200 nanometers, less than or equal to 100 nanometers, less than or equal to 50 nanometers, less than or equal to 10 nanometers, less than or equal to 5 nanometers, less than or equal to 1 nanometer.

In some embodiments, the LNPs have an average mean diameter of between 1 nanometer and 1000 nanometers, between 1 nanometer and 350 nanometers, between 1 nanometer and 300 nanometers, between 1 nanometer and 250 nanometers, between 1 nanometer and 200 nanometers, between 1 nanometer and 150 nanometers, between 1 nanometer and 100 nanometers, or between 1 nanometer and 50 nanometers. In some embodiments, the average mean particle diameter is between 100 nanometers and 900 nanometers, between 200 nanometers and 800 nanometers, between 300 nanometers and 700 nanometers, or between 400 nanometers and 600 nanometers. LNPs with other average mean diameters are also possible, in some embodiments.

In some embodiments, LNPs comprises one or more lipids (e.g., one or more different types of lipids, for example 1, 2, 3, 4, 5, or more different lipids). As used herein, the term lipid refers to any class of organic compounds that are fatty acids or their derivatives and are insoluble in water but soluble in organic solvents (e.g., waxes, fats, oils, hormones, lipid membranes, etc.). In some embodiments, the lipids comprise amphiphilic lipids. In some embodiments, the lipids comprise neutral lipids. In some embodiments, the lipids comprise one or more ionizable lipids. In some embodiments, the one or more ionizable lipids comprises cationic lipids, anionic lipids, or a combination thereof. In some embodiments, the lipids comprise functionalized lipids. In some embodiments, functionalized lipids comprise one or more reactive groups (e.g., amines, carboxyl groups, etc.). In some embodiments, the lipids comprise lipid-conjugates, for example, lipid-polyethylene glycol. In some embodiments, the lipid-conjugates comprise more than one conjugate.

In some embodiments, the lipid is 1,2-dioleoyl-3-trimethylammonium-propane or a derivative thereof (herein “DOTAP”). In some embodiments, the lipid is 4-(dimethylamino)-butanoic acid, (10Z,13Z)-1-(9Z,12Z)-9,12-octadecadien-1-yl-10,13-nonadecadien-1-yl ester or a derivative thereof (e.g., herein “Dlin-MC3-DMA” or “MC3”). In some embodiments, the lipid is Heptadecan-9-yl 8-{(2-hydroxyethyl) [6-oxo-6-(undecyloxy)hexyl]amino}octanoate or a derivative thereof (herein “SM-102”). In some embodiments, the lipid is [(4-Hydroxybutyl)azanediyl]di(hexane-6,1-diyl) bis(2-hexyldecanoate) or a derivative thereof (herein “Alc-0315”). In some embodiments, the lipid is cholesterol or a derivative thereof. In some embodiments, the lipid is (1,2-distearoyl-sn-glycero-3-phosphocholine) or a derivative thereof (herein “DSPC”). In some embodiments, the lipid is 1,2-dimyristoyl-rac-glycero-3-methoxypolyethylene glycol-2000 or a derivative thereof (herein “DMG-PEG-2000”).

In some embodiments, the one or more ionizable lipids comprises MC3 or DOTAP.

In some embodiments, compositions of the disclosure comprise LNP compositions with varying ratios of the one or more lipids. Any suitable ratio known in the art may be used to produce the LNPs disclosed herein. Those familiar with the art, will appreciate that the sum of the ratios from each lipid component cannot exceed 100% (e.g., mole %, weight %, mass %, volume %, etc.). For example, in some embodiments, the percent contribution of each lipid in the LNP composition is greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, or greater than or equal to 100%. In some embodiments, the percent contribution (e.g., mole %) of each lipid in LNP composition is less than or equal to 100%, less than or equal to 95%, less than or equal to 90%, less than or equal to 85%, less than or equal to 80%, less than or equal to 75%, less than or equal to 70%, less than or equal to 65%, less than or equal to 60%, less than or equal to 55%, less than or equal to 50%, less than or equal to 45%, less than or equal to 40%, less than or equal to 35%, less than or equal to 30%, less than or equal to 25%, less than or equal to 20%, less than or equal to 15%, less than or equal to 10%, less than or equal to 5%, less than or equal to 1%.

In some embodiments, the method comprises LNP compositions comprising one or more nucleic acids (e.g., siRNA, mRNA, dsRNA, or miRNA). In some embodiments, the concentration of the one or more nucleic acid in the LNP composition is between about 1 ng/ml and 50 mg/mL. In some embodiments, the concentration of the one or more nucleic acids in the LNP composition is between about 1 mg/mL and 50 mg/mL. In some embodiments, the concentration of the one or more nucleic acids in the LNP composition is between about 500 μg/mL and about 20 mg/mL (e.g., about 500 μg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL). In some embodiments, the concentration of the one or more nucleic acids in the LNP composition is between about 500 μg/mL and 3 mg/mL (e.g., about 500 μg/mL, about 1 mg/mL, about 1.5 mg/mL, about 2 mg/mL, or about 3 mg/mL). In some embodiments, the concentration of the one or more nucleic acids in the LNP composition is between about 1 mg/mL and about 5 mg/mL, for example about 1 mg/mL, about 2 mg/mL, about 3 mg/mL, about 4 mg/mL, or about 5 mg/mL. In some embodiments, the concentration of the one or more nucleic acids in the LNP composition is between about 1.5 mg/mL and 2.5 mg/mL (e.g., about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, or about 2.5 mg/mL).

In some embodiments, an LNP (e.g., LNP composition) comprises a stabilizer. Without wishing to be bound by theory, stabilizers are art recognized compounds capable of improving the stability of any one of the LNPs disclosed herein. In some embodiments, the stabilizer comprises a lipid-polyethylene glycol conjugate. In some embodiments, the stabilizer comprises sucrose. Other stabilizers are also possible in some embodiments.

In some embodiments, the LNPs (e.g., LNP compositions) disclosed herein further comprise an active pharmaceutical ingredient (API). In some embodiments, the LNPs disclosed herein further comprise one or more nucleic acids. In some embodiments, the API comprises one or more nucleic acids. In some embodiments, the one or more nucleic acids comprises RNA. In some embodiments, the RNA is a messenger ribonucleic acid (herein “mRNA”), a small interfering ribonucleic acid (herein “siRNA”), a double stranded ribonucleic acid (herein “dsRNA”), or a micro ribonucleic acid (herein “miRNA”).

In some embodiments, the nucleic acid is a siRNA. siRNAs are art recognized non-coding double-stranded RNAs that operate within the RNA interference pathway. Without being bound by theory, it is believed that they interfere with expression of specific genes with complementary nucleotide sequences by degrading mRNA after transcription, thus preventing translation. In some embodiments, the siRNA is a nucleic acid therapeutic that targets (e.g., silences or inhibits) a gene associated with a human disease or disorder.

In some embodiments, the one or more nucleic acids is a micro-RNA (herein “miRNA”). miRNAs are art recognized single stranded, non-coding RNA molecules involved in RNA silencing and post-transcriptional regulation of gene expression. Without being bound by theory, it is believed that miRNAs base-pair to complementary sequences in mRNAs which allows them to silence the mRNA molecule via cleavage of mRNA strand into two pieces or destabilization of mRNA by shortening its poly(A) tail.

In some embodiments, the one or more nucleic acids are encapsulated within any one of the LNPs disclosed herein. In some embodiments, the one or more nucleic acids may be encapsulated within any structure of any one of the LNPs disclosed herein. For example, in some embodiments, the one or more nucleic acids may be encapsulated within the hydrophobic lipid bilayer of a liposome or a lipoplex. In some embodiments, the one or more nucleic acids may be encapsulated within the hydrophilic core of a liposome or lipoplex. In some embodiments, the one or more nucleic acids may be encapsulated within the lipophilic core of a solid lipid nanoparticle. In some embodiments, the one or more nucleic acids may be encapsulated within one or more revere micelles encapsulated within a micelle. In some embodiments, the one or more nucleic acids may be electrostatically bound to the outside surface of an LNP. Other configurations are also possible in some embodiments.

Aspects of the disclosure relate to compositions (e.g., LNPs) comprising histidine buffers. As used herein, the term “histidine” refers to the essential amino acid comprising an alpha-amino group, a carboxylic acid group, and an imidazole side chain. In some embodiments, the histidine is L-histidine. In some embodiments, the histidine is D-histidine. In some embodiments, the histidine is a combination of L-histidine and D-histidine.

In some embodiments, a histidine buffer has a histidine concentration of between 5 mM and 50 mM. In some embodiments, the histidine concentration is greater than or equal to 5 mM, greater than or equal to 10 mM, greater than or equal to 20 mM, greater than or equal to 30 mM, greater than or equal to 40 mM, or greater than or equal to 50 mM. In some embodiments, the histidine concentration is less than or equal to 50 mM, less than or equal to 40 mM, less than or equal to 30 mM, less than or equal to 20 mM, less than or equal to 10 mM, or less than or equal to 5 mM. In some embodiments, the concentration of the histidine in the histidine buffer is 20 mM. Other combinations are also possible in some embodiments.

In some embodiments, a histidine buffer has a histidine concentration of between 5 mM and 50 mM, between 10 mM and 40 mM, or between 20 mM and 30 mM. Other ranges are also possible in some embodiments.

In some embodiments, a histidine buffer has a histidine concentration of between 5 mM and 25 mM, between 10 mM and 20 mM, between 12 mM and 18 mM, or between 14 mM and 16 mM. In some embodiments, a histidine buffer has a histidine concentration of at least 5 mM, at least 10 mM, at least 20 mM, at least 25 mM, and at least 30 mM. In some embodiments, the histidine buffer has a histidine concentration of 20 mM.

In some embodiments, a histidine buffer has a pH value of between 5.0 to 7.5 (e.g., 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5). In some embodiments, a histidine buffer has a pH value of between 5.8 to 6.5. In some embodiments, the pH is greater than or equal to 5.8, greater than or equal to 5.9, greater than or equal to 6.0, greater than or equal to 6.1, greater than or equal to 6.2, greater than or equal to 6.3, greater than or equal to 6.4, or greater than or equal to 6.5. In some embodiments, the pH is less than or equal to 6.5, less than or equal to 6.4, less than or equal to 6.3, less than or equal to 6.2, less than or equal to 6.1, less than or equal to 6.0, less than or equal to 5.9, or less than or equal to 5.8.

In some embodiments, a histidine buffer has a pH value of between 5.8 to 6.5. In some embodiments, the pH is greater than or equal to 5.8, greater than or equal to 5.9, greater than or equal to 6.0, greater than or equal to 6.1, greater than or equal to 6.2, greater than or equal to 6.3, greater than or equal to 6.4, or greater than or equal to 6.5. In some embodiments, the pH is less than or equal to 6.5, less than or equal to 6.4, less than or equal to 6.3, less than or equal to 6.2, less than or equal to 6.1, less than or equal to 6.0, less than or equal to 5.9, or less than or equal to 5.8.

In some embodiments, a histidine buffer has a pH value of between 5.8 to 6.5. In some embodiments, the histidine buffer has a pH value of between 5.8 to 6.4, between 5.8 and 6.3, between 5.8 and 6.2, between 5.8 and 6.1, between 5.8 and 6.0, or between 5.8 and 5.9. In some embodiments, the histidine buffer has a pH value of between 5.9 and 6.4, between 6.0 and 6.3, or between 6.1 and 6.2. In some embodiments, the histidine buffer has a pH value of between 5.9 and 6.4, between 6.0 and 6.4, between 6.1 and 6.4, between 6.2 and 6.4, or between 6.3 and 6.4. In some embodiments, the histidine buffer has a pH of about 6.0.

In some embodiments, a histidine buffer comprises one or more salts. In some embodiments, the histidine buffer comprises sodium chloride. In some embodiments, the concentration of the one or more salts is between 10 mM and 300 mM. In some embodiments, the concentration of the one or more salts is greater than or equal to 10 mM, greater than or equal to 20 mM, greater than or equal to 40 mM, greater than or equal to 80 mM greater than or equal to 100 mM greater than or equal to 120 mM greater than or equal to 140 mM greater than or equal to 160 mM greater than or equal to 180 mM greater than or equal to 200 mM, greater than or equal to 220 mM, greater than or equal to 240 mM, greater than or equal to 260 mM, greater than or equal to 280 mM, greater than or equal to 300 mM. In some embodiments, the concentration of the one or more salts in the histidine buffer is less than or equal to 300 mM, less than or equal to 280 mM, less than or equal to 260 mM, less than or equal to 240 mM, less than or equal to 220 mM, less than or equal to 200 mM, less than or equal to 180 mM, less than or equal to 160 mM, less than or equal to 140 mM, less than or equal to 120 mM, less than or equal to 100 mM, less than or equal to 80 mM, less than or equal to 40 mM, less than or equal to 20 mM, less than or equal to 10 mM.

In some embodiments, the concentration of the one or more salts is between 10 mM and 300 mM, between 20 mM and 280 mM, between 40 mM and 260 mM, between 80 mM and 240 mM, between 100 mM and 220 mM, between 120 mM and 200 mM, and between 140 mM and 180 mM. Other concentrations are also possible in other ranges and/or combinations in some embodiments.

In some embodiments, a histidine buffer comprises one or more non-ionic excipients. Examples of non-ionic excipients include sugars (e.g., sucrose), alcohols, polysorbates, etc. In some embodiments, the concentration of the one or more non-ionic excipients is between 10 mM and 300 mM. In some embodiments, the concentration of the one or more non-ionic excipients is greater than or equal to 10 mM, greater than or equal to 20 mM, greater than or equal to 40 mM, greater than or equal to 80 mM greater than or equal to 100 mM greater than or equal to 120 mM greater than or equal to 140 mM greater than or equal to 160 mM greater than or equal to 180 mM greater than or equal to 200 mM, greater than or equal to 220 mM, greater than or equal to 240 mM, greater than or equal to 260 mM, greater than or equal to 280 mM, greater than or equal to 300 mM. In some embodiments, the concentration of the one or more non-ionic excipients in the histidine buffer is less than or equal to 300 mM, less than or equal to 280 mM, less than or equal to 260 mM, less than or equal to 240 mM, less than or equal to 220 mM, less than or equal to 200 mM, less than or equal to 180 mM, less than or equal to 160 mM, less than or equal to 140 mM, less than or equal to 120 mM, less than or equal to 100 mM, less than or equal to 80 mM, less than or equal to 40 mM, less than or equal to 20 mM, less than or equal to 10 mM.

Additional aspects of the present disclosure relate to storage of any one of the compositions described herein. For example, in some embodiments, the compositions are stored in a container such as a cartridge, prefilled syringe, or vial. In some embodiments, the container is a glass vial. In some embodiments, the container is a polycarbonate vial. It is known in the art, that all parenteral drugs must be stored in Type 1 glass (e.g., USP <660>, EP 3.2.1, ASTM E438) and meet requirements for hydrolytic resistance. However, the composition of the glass may vary significantly from manufacturer to manufacturer. Thus, in some embodiments, the vials may be obtained from a number of different manufacturers. In some embodiments, the vial comprises cyclic olefin polymer (herein “COP”). In some embodiments, the vial comprises glass. In some embodiments, the glass vial is a Corning Valor® glass vial. In some embodiments, the glass vial is a Schott BT5933 glass vial. In some embodiments, the glass vial is a Gerresheimer BT5974 glass vial. In some embodiments, the glass vial may comprise a coating. Exemplary coatings include, but are not limited to, ammonium sulfate, quartz (e.g., SiOx), SiO2, and the like. In some embodiments, the vials may have a coefficient of expansion (herein “COE”) of 33 or 51, although other COEs are also contemplated herein. In some embodiments, the vials may have a volume of between 1 mL and 20 mL. In some embodiments, the vial has a volume of greater than or equal to 1 mL, greater than or equal to 5 mL, greater than or equal to 10 mL, greater than or equal to 15 mL, or greater than or equal to 20 mL. In some embodiments, the vial has less than or equal to 20 mL, less than or equal to 15 mL, less than or equal to 10 mL, less than or equal to 5 mL, or less than or equal to 1 mL.

In some embodiments, the vials comprise a pharmaceutical rubber stopper or a cap. Any suitable pharmaceutical rubber stopper or cap known in the art may be used herein. In some embodiments, the pharmaceutical rubber stopper or cap is provided with the vials (e.g., a Corning Valor® vial is supplied with its own rubber stopper). In some embodiments, a pharmaceutical rubber stopper is a VS5558 Serum stopper.

In some embodiments, the compositions are stored at a temperature above 4° C. In some embodiments, the compositions are stored at a temperature between about 5° C. and about 30° C., between about 5° C. and about 70° C., between about 10° C. and about 60° C., between about 15° C. and about 55° C., between about 20° C. and about 50° C., between about 25° C. and about 45° C., and between about 30° C. and 40° C.

In some embodiments, the compositions are not cooled to less than or equal to 4° C. (e.g., temperature of most commercial refrigerators) or less than or equal to −20° C. (e.g., temperature of most commercial freezers).

In some embodiments, the methods comprise storing the compositions are stored at a temperature between 5° C. and 25° C. In some embodiments, the compositions are stored at a temperature between 5° C. and 30° C. In some embodiments, the temperature is greater than or equal to 5° C., greater than or equal to 10° C., greater than or equal to 15° C., greater than or equal to 20° C., greater than or equal to 25° C., greater than or equal to 30° C., greater than or equal to 35° C., greater than or equal to 40° C., greater than or equal to 45° C., greater than or equal to 50° C., greater than or equal to 55° C., greater than or equal to 60° C., greater than or equal to 65° C., or greater than or equal to 70° C. In some embodiments, the temperature is less than or equal to 70° C., less than or equal to 65° C., less than or equal to 60° C., less than or equal to 55° C., less than or equal to 50° C., less than or equal to 45° C., less than or equal to 40° C., less than or equal to 35° C., less than or equal to 30° C., less than or equal to 25° C., less than or equal to 20° C., less than or equal to 10° C., or less than or equal to 5° C. Other combinations are also possible in some embodiments (e.g., greater than or equal to 5° C. and less than or equal to 30° C. or greater than or equal to 5° C. and less than or equal to 70° C.).

Aspects of the disclosure relate to compositions comprising LNPs comprising one or more ionizable lipids and histidine buffers disclosed elsewhere herein. In some embodiments, the one or more ionizable lipids is MC3 or DOTAP. In some embodiments, the histidine buffer has a concentration ranging from about 5 mM to 25 mM of histidine. In some embodiments, the histidine buffer has a pH ranging from about 5.8 to about 7.5.

In some embodiments, compositions comprising histidine-buffered LNP compositions have at least a 200% reduction in the concentration of subvisible particles, relative to LNPs stored in phosphate buffer after 4 weeks of storage at 25° C. As used herein, the term “subvisible particle” refers to particles that are too large for analysis by size exclusion chromatography (SEC) (e.g., ~>0.1 μm), but too small to be visible to the unaided eye (e.g., <100 μm). In some embodiments, subvisible particles range in size from about 10 μm to about 25 μm,

In some embodiments, the compositions comprising histidine-buffered LNP compositions have at least a 10%, at least 20%, at least 30%, at least 40%, at least 50%, at least 60%, at least 70%, at least 80%, at least 90%, at least 100%, at least 120%, at least 140%, at least 160%, at least 180%, or at least 200% reduction in the concentration of subvisible particles, relative to LNPs stored in phosphate buffer after 4 weeks of storage at 25° C.

In some embodiments, compositions comprising histidine-buffered LNP compositions have an API concentration of at least 0.11 mg/mL or an encapsulation efficiency of at least 95%, relative to LNPs stored in phosphate buffer after 4 weeks of storage at 25° C. In some embodiments, the compositions comprising histidine-buffered LNP compositions have an siRNA concentration of at least 0.11 mg/mL or an encapsulation efficiency of at least 95%, relative to LNPs stored in phosphate buffer after 4 weeks of storage at 25° C.

In some embodiments, the compositions comprising histidine-buffered LNP compositions undergo between 0.1% and 2% hydrolysis after 4 weeks of storage at 25° C. In some embodiments, the compositions comprising histidine-buffered LNP compositions undergo between 0.1% and 1% oxidation after 4 weeks of storage at 25° C. In some embodiments, the compositions comprising histidine-buffered LNP compositions contain between 95% and 98.5% intact LNPs after 4 weeks of storage at 25° C.

### Methods

Aspects of the disclosure relate to one or more methods for producing LNP compositions disclosed herein. Exemplary LNPs include, but are not limited to, micelles, liposomes, lipoplexes, and solid lipid nanoparticles or derivatives thereof. LNPs may have any morphology and structure known in the art. For example, in some embodiments, LNPs are nanospheres, nanorods, nanochains, nanostars, nanoflowers, nanoreefs, nanowhiskers, nanofibers, and nanoboxes. In some embodiments, other morphologies and structures are also possible.

In some embodiments, the method comprises LNP compositions with varying ratios of one or more lipids. Any suitable ratio known in the art may be used to produce the LNPs disclosed herein. Those familiar with the art, will appreciate that the sum of the percent contribution from each lipid component cannot exceed 100% (e.g., mole %, weight %, mass %, volume %, etc.). For example, in some embodiments, the percent contribution of each lipid in the LNP composition is greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, or greater than or equal to 100%. In some embodiments, the percent contribution (e.g., mole %) of each lipid in LNP composition is less than or equal to 100%, less than or equal to 95%, less than or equal to 90%, less than or equal to 85%, less than or equal to 80%, less than or equal to 75%, less than or equal to 70%, less than or equal to 65%, less than or equal to 60%, less than or equal to 55%, less than or equal to 50%, less than or equal to 45%, less than or equal to 40%, less than or equal to 35%, less than or equal to 30%, less than or equal to 25%, less than or equal to 20%, less than or equal to 15%, less than or equal to 10%, less than or equal to 5%, less than or equal to 1%.

In some embodiments, the methods comprise formulating the LNP compositions using a histidine buffer. In some embodiments, a histidine buffer has a histidine concentration of between 5 mM and 50 mM. In some embodiments, the histidine concentration is greater than or equal to 5 mM, greater than or equal to 10 mM, greater than or equal to 20 mM, greater than or equal to 30 mM, greater than or equal to 40 mM, or greater than or equal to 50 mM. In some embodiments, the histidine concentration is less than or equal to 50 mM, less than or equal to 40 mM, less than or equal to 30 mM, less than or equal to 20 mM, less than or equal to 10 mM, or less than or equal to 5 mM. In some embodiments, the concentration of the histidine in the histidine buffer is 20 mM. Other combinations are also possible in some embodiments.

In some embodiments, a histidine buffer has a histidine concentration of between 5 mM and 25 mM, between 10 mM and 20 mM, between 12 mM and 18 mM, or between 14 mM and 16 mM. In some embodiments, a histidine buffer has a histidine concentration of at least 5 mM, at least 10 mM, at least 20 mM, at least 25 mM, and at least 30 mM. In some embodiments, the histidine buffer has a histidine concentration of 20 mM.

In some embodiments, a histidine buffer has a histidine concentration of between 5 mM and 50 mM, between 10 mM and 40 mM, or between 20 mM and 30 mM. Other ranges are also possible in some embodiments.

In some embodiments, a histidine buffer has a pH value of between 5.0 to 6.5 (e.g., 5.0, 5.1, 5.2, 5.3, 5.4, 5.5, 5.6, 5.7, 5.8, 5.9, 6.0, 6.1, 6.2, 6.3, 6.4, or 7.5). In some embodiments, a histidine buffer has a pH value of between 5.8 to 6.5. In some embodiments, the pH is greater than or equal to 5.8, greater than or equal to 5.9, greater than or equal to 6.0, greater than or equal to 6.1, greater than or equal to 6.2, greater than or equal to 6.3, greater than or equal to 6.4, or greater than or equal to 6.5. In some embodiments, the pH is less than or equal to 6.5, less than or equal to 6.4, less than or equal to 6.3, less than or equal to 6.2, less than or equal to 6.1, less than or equal to 6.0, less than or equal to 5.9, or less than or equal to 5.8.

In some embodiments, a histidine buffer has a pH value of between 5.8 to 6.5. In some embodiments, the pH is greater than or equal to 5.8, greater than or equal to 5.9, greater than or equal to 6.0, greater than or equal to 6.1, greater than or equal to 6.2, greater than or equal to 6.3, greater than or equal to 6.4, or greater than or equal to 6.5. In some embodiments, the pH is less than or equal to 6.5, less than or equal to 6.4, less than or equal to 6.3, less than or equal to 6.2, less than or equal to 6.1, less than or equal to 6.0, less than or equal to 5.9, or less than or equal to 5.8.

In some embodiments, a histidine buffer has a pH value of between 5.8 to 6.5. In some embodiments, the histidine buffer has a pH value of between 5.8 to 6.4, between 5.8 and 6.3, between 5.8 and 6.2, between 5.8 and 6.1, between 5.8 and 6.0, or between 5.8 and 5.9. In some embodiments, the histidine buffer has a pH value of between 5.9 and 6.4, between 6.0 and 6.3, or between 6.1 and 6.2. In some embodiments, the histidine buffer has a pH value of between 5.9 and 6.4, between 6.0 and 6.4, between 6.1 and 6.4, between 6.2 and 6.4, or between 6.3 and 6.4. In some embodiments, the histidine buffer has a pH of 6.0.

In some embodiments, a histidine buffer comprises one or more salts. In some embodiments, the histidine buffer comprises sodium chloride. In some embodiments, the concentration of the one or more salts is between 10 mM and 300 mM. In some embodiments, the concentration of the one or more salts is greater than or equal to 10 mM, greater than or equal to 20 mM, greater than or equal to 40 mM, greater than or equal to 80 mM greater than or equal to 100 mM greater than or equal to 120 mM greater than or equal to 140 mM greater than or equal to 160 mM greater than or equal to 180 mM greater than or equal to 200 mM, greater than or equal to 220 mM, greater than or equal to 240 mM, greater than or equal to 260 mM, greater than or equal to 280 mM, greater than or equal to 300 mM. In some embodiments, the concentration of the one or more salts in the histidine buffer is less than or equal to 300 mM, less than or equal to 280 mM, less than or equal to 260 mM, less than or equal to 240 mM, less than or equal to 220 mM, less than or equal to 200 mM, less than or equal to 180 mM, less than or equal to 160 mM, less than or equal to 140 mM, less than or equal to 120 mM, less than or equal to 100 mM, less than or equal to 80 mM, less than or equal to 40 mM, less than or equal to 20 mM, less than or equal to 10 mM.

In some embodiments, the concentration of the one or more salts is between 10 mM and 300 mM, between 20 mM and 280 mM, between 40 mM and 260 mM, between 80 mM and 240 mM, between 100 mM and 220 mM, between 120 mM and 200 mM, and between 140 mM and 180 mM. Other concentrations are also possible in other ranges and/or combinations in some embodiments.

Aspects of the disclosure relate to methods for improving the chemical stability of a LNP pharmaceutical compositions, such as, for example, the compositions disclosed herein. In some embodiments, the method comprises obtaining an LNP pharmaceutical composition comprising a non-histidine buffer. The term “non-histidine” buffer as used herein refers to any buffer that does not contain the essential amino acid histidine. In some embodiments, the non-histidine buffer comprises a phosphate buffer (e.g., phosphate buffered saline). In some embodiments, the non-histidine buffer comprises phosphate buffered saline (PBS). However, other non-histidine buffers are also possible, according to some embodiments. For example, in some embodiments, the non-histidine buffer is a bicarbonate buffer, a HEPES buffer, a MOPS buffer, a PBST buffer, a TBST buffer, a TE buffer, a TEN buffer or the like. Any method of preparation known in the art may be used to prepare any non-histidine buffers contemplated herein, such as those described in Stoll et al., “Buffers: Principles and practice.” Meth. Enzymol. 1990. 182, 24-38.

In some embodiments, the concentration of the conjugate acid-base pair used to create the non-histidine buffer is between 5 mM and 50 mM. In some embodiments, the concentration is greater than or equal to 5 mM, greater than or equal to 10 mM, greater than or equal to 20 mM, greater than or equal to 30 mM, greater than or equal to 40 mM, or greater than or equal to 50 mM. In some embodiments, the concentration is less than or equal to 50 mM, less than or equal to 40 mM, less than or equal to 30 mM, less than or equal to 20 mM, less than or equal to 10 mM, or less than or equal to 5 mM. Other combinations are also possible in some embodiments.

In some embodiments, the concentration of the conjugate acid-base pair used to create the non-histidine buffer is between 5 mM and 50 mM, between 10 mM and 40 mM, or between 20 mM and 30 mM. Other ranges are also possible in some embodiments.

In some embodiments, a non-histidine buffer has a pH value of between 5.8 to 6.5. In some embodiments, the pH is greater than or equal to 5.8, greater than or equal to 5.9, greater than or equal to 6.0, greater than or equal to 6.1, greater than or equal to 6.2, greater than or equal to 6.3, greater than or equal to 6.4, or greater than or equal to 6.5. In some embodiments, the pH is less than or equal to 6.5, less than or equal to 6.4, less than or equal to 6.3, less than or equal to 6.2, less than or equal to 6.1, less than or equal to 6.0, less than or equal to 5.9, or less than or equal to 5.8.

In some embodiments, a non-histidine buffer has a pH value of between 5.8 to 6.5. In some embodiments, the non-histidine buffer has a pH value of between 5.8 to 6.4, between 5.8 and 6.3, between 5.8 and 6.2, between 5.8 and 6.1, between 5.8 and 6.0, or between 5.8 and 5.9. In some embodiments, the non-histidine buffer has a pH value of between 5.9 and 6.4, between 6.0 and 6.3, or between 6.1 and 6.2. In some embodiments, the non-histidine buffer has a pH value of between 5.9 and 6.4, between 6.0 and 6.4, between 6.1 and 6.4, between 6.2 and 6.4, or between 6.3 and 6.4.

In some embodiments, the non-histidine buffer comprises one or more salts. In some embodiments, the non-histidine buffer comprises sodium chloride. In some embodiments, the concentration of the one or more salts is between 10 mM and 300 mM. In some embodiments, the concentration of the one or more salts is greater than or equal to 10 mM, greater than or equal to 20 mM, greater than or equal to 40 mM, greater than or equal to 80 mM greater than or equal to 100 mM greater than or equal to 120 mM greater than or equal to 140 mM greater than or equal to 160 mM greater than or equal to 180 mM greater than or equal to 200 mM, greater than or equal to 220 mM, greater than or equal to 240 mM, greater than or equal to 260 mM, greater than or equal to 280 mM, greater than or equal to 300 mM. In some embodiments, the concentration of the one or more salts in the non-histidine buffer is less than or equal to 300 mM, less than or equal to 280 mM, less than or equal to 260 mM, less than or equal to 240 mM, less than or equal to 220 mM, less than or equal to 200 mM, less than or equal to 180 mM, less than or equal to 160 mM, less than or equal to 140 mM, less than or equal to 120 mM, less than or equal to 100 mM, less than or equal to 80 mM, less than or equal to 40 mM, less than or equal to 20 mM, less than or equal to 10 mM.

In some embodiments, the non-histidine buffer comprises salts at a concentration of between 10 mM and 300 mM, between 20 mM and 280 mM, between 40 mM and 260 mM, between 80 mM and 240 mM, between 100 mM and 220 mM, between 120 mM and 200 mM, and between 140 mM and 180 mM. Other concentrations are also possible in other ranges and/or combinations in some embodiments.

In some embodiments, methods described herein comprise performing a buffer exchange reaction to replace a non-histidine buffer with a histidine buffer to obtain a histidine-buffered LNP pharmaceutical composition. Any method for exchanging buffers known in the art may be used in for the buffer exchange reaction. Exemplary embodiments, include but are not limited to, dialysis, desalting, and diafiltration. In some embodiments, diafiltration comprises performing tangential flow filtration (TFF).

In some embodiments, the buffer exchange reaction is performed using dialysis. Without wishing to be bound by theory, dialysis separates small molecules from large molecules by allowing diffusion of only the small molecules through selectively permeable membranes. The solution to be dialyzed (e.g., non-histidine containing LNP composition) is placed in a sealed dialysis membrane, of a particular molecular weight cutoff, and immersed in a selected buffer (e.g., histidine containing buffer). The non-histidine buffer molecules diffuse out of the dialysis bag while the histidine buffer molecules diffuse into the bag (e.g., along their respective diffusion gradients). Once the solution reaches equilibrium, the buffer exchange reaction stops. To restart the buffer exchange reaction, the dialysate must be replaced with fresh histidine buffer to re-establish the concentration gradients. This procedure is repeated until the non-histidine buffer is completely removed from the dialysis membrane.

In some embodiments, the buffer exchange reaction is performed using desalting. Without wishing to be bound by theory, desalting column are based on gel filtration chromatography techniques in which a solution containing the buffer to be exchanged (e.g., non-histidine buffer) is added to a porous resin. Larger molecules in the solution (e.g., LNPs) flow around the porous resin via the void spaces, whereas smaller molecules (e.g., non-histidine salts) enter into the pores of the porous resin. By passing sample through a column resin bed of sufficient length and volume, macromolecules (e.g., LNPs) can be fully separated from small molecules that travel a greater distance through the pores of the resin bed. Varying the maximum effective pore size is known in the art to be the primary determinant of the size of molecules that can be separated for that particular resin (e.g., also referred to as the molecular weight cut off, MWCO). In some embodiments, any suitable MWCO known in the art may be used to perform the buffer exchange reaction as disclosed herein.

In some embodiments, desalting columns may perform the buffer exchange reaction either directly or indirectly. For example, in some embodiments, the desalting column may be used to separate a composition comprising LNPs and a non-histidine buffer into LNPs in water and non-histidine buffers in water. In this case, the desired buffer salts may be added directly to the aqueous solution of LNPs to yield the final composition comprising LNPs in a histidine buffer. Alternatively, the desalting column may be pre-equilibrated with the desired final buffer (e.g., histidine buffer). In this case, the LNPs mix with the histidine buffer as they pass through the void space and elute from the column.

In some embodiments, desalting may be performed using a variety of formats, for example, chromatography columns, gravity-flow columns, chromatography cartridges, centrifuge columns, and centrifuge plates. Other formats are also possible is some embodiments.

In some embodiments, the buffer exchange reaction is performed using diafiltration. Without wishing to be bound by theory, diafiltration is a process that simultaneously dilutes and filters a solution. In this way, a composition (e.g., LNPs composition comprising a non-histidine buffer) may be filtered to remove small molecules (e.g., salts) while continuously diluted with a desired diluent (e.g., a histidine buffer). As described elsewhere herein, the MWCO of the filters (e.g., resins, dialysis tubing, etc.) play an important role in determining what salts may be removed and which are retained. Thus, in some embodiments, any suitable MWCO known in the art may be used to perform the buffer exchange reaction as disclosed herein.

In some embodiments, the methods comprise storing the histidine-buffered LNP pharmaceutical composition to improve the stability of the composition. Without wishing to be bound by theory, it is believed that various lipid components of the LNPs may undergo degradation via ester hydrolysis or oxidation of unsaturated bonds when stored in non-histidine buffers (e.g., phosphate buffer) for prolonged times and at different temperatures. It has now been discovered that storing the LNP pharmaceutical compositions in a histidine buffer reduces the degradation (e.g., oxidation and/or hydrolysis) of one or more lipid components, relative to LNP pharmaceutical compositions stored in a non-histidine buffer (e.g., phosphate buffer).

In some embodiments, the methods comprise storing LNP pharmaceutical compositions in vials. In some embodiments, the compositions are stored in a container such as a cartridge, prefilled syringe, or vial. In some embodiments, the container is a glass vial. In some embodiments, the container is a polycarbonate vial. It is known in the art, that all parenteral drugs must be stored in Type 1 glass (e.g., USP <660>, EP 3.2.1, ASTM E438) and meet requirements for hydrolytic resistance. However, the composition of the glass may vary significantly from manufacturer to manufacturer. Thus, in some embodiments, the vials may be obtained from a number of different manufacturers. In some embodiments, the vial comprises cyclic olefin polymer (herein “COP”). In some embodiments, the vial comprises glass. In some embodiments, the glass vial is a Corning Valor® glass vial. In some embodiments, the glass vial is a Schott BT5933 glass vial. In some embodiments, the glass vial is a Gerresheimer BT5974 glass vial. In some embodiments, the glass vial may comprise a coating. Exemplary coatings include, but are not limited to, ammonium sulfate, quartz (e.g., SiOx), SiO2, and the like. In some embodiments, the vials may have a coefficient of expansion (herein “COE”) of 33 or 51, although other COEs are also contemplated herein. In some embodiments, the vials may have a volume of between 1 mL and 20 mL. In some embodiments, the vial has a volume of greater than or equal to 1 mL, greater than or equal to 5 mL, greater than or equal to 10 mL, greater than or equal to 15 mL, or greater than or equal to 20 mL. In some embodiments, the vial has less than or equal to 20 mL, less than or equal to 15 mL, less than or equal to 10 mL, less than or equal to 5 mL, or less than or equal to 1 mL.

In some embodiments, the vials comprise a pharmaceutical rubber stopper or a cap. Any suitable pharmaceutical rubber stopper or cap known in the art may be used herein. In some embodiments, the pharmaceutical rubber stopper or cap is provided with the vials (e.g., a Corning Valor® vial is supplied with its own rubber stopper). In some embodiments, a pharmaceutical rubber stopper is a VS5558 Serum stopper.

In some embodiments, the methods comprise storing the LNP pharmaceutical compositions in a histidine buffer at a temperature above 4° C. In some embodiments, the compositions are stored at a temperature between about 5° C. and about 30° C., between about 5° C. and about 70° C., between about 10° C. and about 60° C., between about 15° C. and about 55° C., between about 20° C. and about 50° C., between about 25° C. and about 45° C., and between about 30° C. and 40° C.

In some embodiments, the compositions are not cooled to less than or equal to 4° C. (e.g., temperature of most commercial refrigerators) or less than or equal to −20° C. (e.g., temperature of most commercial freezers). In some embodiments, the compositions are frozen (e.g., stored below 0° C., for example at −20° C. or −70° C.) and subsequently thawed.

In some embodiments, the methods comprise storing the compositions are stored at a temperature between 5° C. and 25° C. In some embodiments, the compositions are stored at a temperature between 5° C. and 30° C. In some embodiments, the temperature is greater than or equal to 5° C., greater than or equal to 10° C., greater than or equal to 15° C., greater than or equal to 20° C., greater than or equal to 25° C., greater than or equal to 30° C., greater than or equal to 35° C., greater than or equal to 40° C., greater than or equal to 45° C., greater than or equal to 50° C., greater than or equal to 55° C., greater than or equal to 60° C., greater than or equal to 65° C., or greater than or equal to 70° C. In some embodiments, the temperature is less than or equal to 70° C., less than or equal to 65° C., less than or equal to 60° C., less than or equal to 55° C., less than or equal to 50° C., less than or equal to 45° C., less than or equal to 40° C., less than or equal to 35° C., less than or equal to 30° C., less than or equal to 25° C., less than or equal to 20° C., less than or equal to 10° C., or less than or equal to 5° C. Other combinations are also possible in some embodiments (e.g., greater than or equal to 5° C. and less than or equal to 30° C. or greater than or equal to 5° C. and less than or equal to 70° C.).

In some embodiments, the methods comprise adjusting the pH of a histidine buffer during storage to a pH value of between 5.8 to 6.5. In some embodiments, the pH is greater than or equal to 5.8, greater than or equal to 5.9, greater than or equal to 6.0, greater than or equal to 6.1, greater than or equal to 6.2, greater than or equal to 6.3, greater than or equal to 6.4, or greater than or equal to 6.5. In some embodiments, the pH is less than or equal to 6.5, less than or equal to 6.4, less than or equal to 6.3, less than or equal to 6.2, less than or equal to 6.1, less than or equal to 6.0, less than or equal to 5.9, or less than or equal to 5.8.

In some embodiments, the methods comprise adjusting the pH of a histidine buffer during storage to a pH value of between 5.8 to 6.5. In some embodiments, the histidine buffer has a pH value of between 5.8 to 6.4, between 5.8 and 6.3, between 5.8 and 6.2, between 5.8 and 6.1, between 5.8 and 6.0, or between 5.8 and 5.9. In some embodiments, the histidine buffer has a pH value of between 5.9 and 6.4, between 6.0 and 6.3, or between 6.1 and 6.2. In some embodiments, the histidine buffer has a pH value of between 5.9 and 6.4, between 6.0 and 6.4, between 6.1 and 6.4, between 6.2 and 6.4, or between 6.3 and 6.4.

In some embodiments, the methods comprise storing a histidine-buffered LNP pharmaceutical composition for 4 weeks at 25° C., wherein storing the composition increases the number of intact LNPs in a histidine-buffered LNP pharmaceutical composition, relative to a LNP pharmaceutical composition stored in a phosphate buffer. For example, in some embodiments, the percent increase of intact LNPs in the histidine-buffered LNP pharmaceutical compositions, relative to LNP compositions stored in a phosphate buffer, is between about 0.5% to 14%. In some embodiments, the percent increase is greater than or equal to 0.5%, greater than or equal to 1%, greater than or equal to 2%, greater than or equal to 3%, greater than or equal to 4%, greater than or equal to 5%, greater than or equal to 6%, greater than or equal to 7%, greater than or equal to 8%, greater than or equal to 9%, greater than or equal to 10%, greater than or equal to 11%, greater than or equal to 12%, greater than or equal to 13%, or greater than or equal to 14%, relative to LNP compositions stored in a phosphate buffer after 4 weeks at 25° C. In some embodiments, the percent increase is less than or equal to 14%, less than or equal to 14%, less than or equal to 13%, less than or equal to 12%, less than or equal to 11%, less than or equal to 10%, less than or equal to 9%, less than or equal to 8%, less than or equal to 7%, less than or equal to 6%, less than or equal to 5%, less than or equal to 4%, less than or equal to 3%, less than or equal to 2%, less than or equal to 1%, less than or equal to 0.5%, relative to LNP compositions stored in a phosphate buffer after 4 week at 25° C.

In some embodiments, the percent increase of intact LNPs in a histidine-buffered LNP pharmaceutical composition, relative to a LNP composition stored in a phosphate buffer, is between 0.5% and 14%, between 1% and 12%, between 2% and 10%, between 3% and 8%, and between 4% and 6%.

In some embodiments, the methods comprise storing a histidine-buffered LNP pharmaceutical composition, wherein storing the composition reduces the percent degradation (e.g., hydrolysis and/or oxidation) of one or more lipid components, relative to a LNP compositions stored in a phosphate buffer. In some embodiments, the percent decrease in degradation is between 40% and 85%, relative to LNP compositions stored in phosphate buffers. In some embodiments, the percent decrease in degradation is greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, or greater than or equal to 85%, relative to LNP compositions stored in phosphate buffers. In some embodiments, the percent degradation is less than or equal to 85%, less than or equal to 80%, less than or equal to 75%, less than or equal to 70%, less than or equal to 65%, less than or equal to 60%, less than or equal to 55%, less than or equal to 50%, less than or equal to 45%, less than or equal to 40%, relative to LNP compositions stored in phosphate buffers.

In some embodiments, the methods comprise storing a histidine-buffered LNP pharmaceutical composition, wherein storing the composition reduces the percent increase in the percent hydrolysis by at least 85%, relative to a LNP compositions stored in a phosphate buffer after 4 weeks at 25° C.

In some embodiments, percent increase in the percent hydrolysis is reduced by at least 5%, at least 10%, at least 20%, at least 30%, at least 40%, at least 50%, at least 60%, at least 70%, at least 80%, or at least 85%, relative to a LNP compositions stored in a phosphate buffer after 4 weeks at 25° C.

In some embodiments, the methods comprise storing a histidine-buffered LNP pharmaceutical composition, wherein storing the composition reduces the percent increase in the percent oxidation by at least 33%, relative to a LNP compositions stored in a phosphate buffer.

In some embodiments, percent increase in the percent oxidation is reduced by at least 5%, at least 15%, at least 20%, at least 25%, at least 30%, at least 31%, at least 32%, or at least 33%, relative to a LNP compositions stored in a phosphate buffer after 4 weeks at 25° C. In some embodiments, the methods disclosed herein increase the colloidal stability of histidine buffered LNP pharmaceutical compositions relative to LNP pharmaceutical compositions stored in phosphate buffers. Without wishing to be bound by theory, it is believed that colloidal instability results in LNP aggregation which increases the LNP mean particle diameter, for example, as measured using dynamic light scattering and/or increases the turbidity of the compositions, for example, as measured using transmitted light.

In some embodiments, storing the histidine-buffered LNP pharmaceutical composition decreases the percent increase in the mean particle diameter of the composition, relative to LNP compositions stored in a phosphate buffer after 4 weeks at 25° C. In some embodiments, storing the compositions decreases the percent increase in mean particle diameter by between 70% and 100%. In some embodiments, storing the compositions decreases the percent increase in mean particle diameter by greater than or equal to 70%, greater than or equal to 75%, greater than or equal to 80%, greater than or equal to 85%, greater than or equal to 90%, greater than or equal to 95%, or greater than or equal to 100%, relative to LNP compositions stored in a phosphate buffer after 4 weeks at 25° C. In some embodiments, storing the compositions decreases the percent increase in mean particle diameter by less than or equal to 100%, less than or equal to 95%, less than or equal to 90%, less than or equal to 85%, less than or equal to 80%, less than or equal to 75%, or less than or equal to 70%, relative to LNP compositions stored in a phosphate buffer after 4 weeks at 25° C.

In some embodiments, storing the histidine-buffered LNP pharmaceutical compositions decreases the percent increase in the mean particle diameter of the composition by a between about 70% and 100%, between about 75% and 95%, or between about 80% and 90%.

In some embodiments, storing the histidine-buffered LNP pharmaceutical compositions decreases turbidity of the composition by between 1% and 99%, relative to LNP compositions stored in a phosphate buffer after 4 weeks at 25° C. In some embodiments, the percent increase in turbidity of the composition is decreased by greater than or equal to 5%, greater than or equal to 10%, greater than or equal to 50%, greater than or equal to 75%, greater than or equal to 90%, relative to LNP compositions stored in a phosphate buffer after 4 weeks at 25° C. In some embodiments, the percent increase in turbidity of the composition is less than or equal to 5%, less than or equal to 10%, less than or equal to 50%, less than or equal to 75%, or less than or equal to 90%, relative to LNP compositions stored in a phosphate buffer after 4 weeks at 25° C.

In some embodiments, storing the histidine-buffered LNP pharmaceutical compositions decreases turbidity of the composition by between about 5% and 10%, between about 25% and 75%, or between about 50% and 90% relative to LNP compositions stored in a phosphate buffer after 4 weeks at 25° C.

Aspects of the present disclosure further relate to methods for improving the encapsulation efficiency of an API within one or more of the histidine-buffered LNP compositions disclosed herein. Methods of encapsulating APIs (e.g., nucleic acids such as siRNA, miRNA, dsRNA, mRNA, etc.) are generally known, for example as described by Mendonca et al. Drug Discov Today. 2023 March; 28(3):103505.

In some embodiments, the method comprises preparing a first lipid composition in any one of the histidine buffers disclosed herein. Any suitable lipid composition known in the art may be used to produce the first lipid composition. For example, in some embodiments, first lipid composition comprises Dlin-MC3-DMA, cholesterol, DSPC, and DMG-PEG-2k. In some embodiments, the first lipid composition comprises SM-102, cholesterol, DSPC, and DMG-PEG-2k. In some embodiments, first lipid composition comprises Alc-0315, cholesterol, DSPC, and DMG-PEG-2k.

In some embodiments, the lipids in the first lipid composition are mixed at various ratios. Any suitable ratio known in the art may be used to produce the LNPs disclosed herein. Those familiar with the art, will appreciate that the sum of the percent contribution from each lipid component cannot exceed 100% (e.g., mole %, weight %, mass %, volume %, etc.). For example, in some embodiments, the mole percent of each lipid in the first lipid composition is greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, or greater than or equal to 100%. In some embodiments, the mole percent of each lipid in the first lipid composition is less than or equal to 100%, less than or equal to 95%, less than or equal to 90%, less than or equal to 85%, less than or equal to 80%, less than or equal to 75%, less than or equal to 70%, less than or equal to 65%, less than or equal to 60%, less than or equal to 55%, less than or equal to 50%, less than or equal to 45%, less than or equal to 40%, less than or equal to 35%, less than or equal to 30%, less than or equal to 25%, less than or equal to 20%, less than or equal to 15%, less than or equal to 10%, less than or equal to 5%, less than or equal to 1%.

In some embodiments, the methods comprise preparing a second lipid composition. Any suitable lipid composition known in the art may be used to produce the second lipid composition. In some embodiments, the second lipid composition comprises DOTAP. In some embodiments, the second lipid composition comprises Dlin-MC3-DMA. Other compositions are also possible in some embodiments.

In some embodiments, the first and/or second lipid composition has a lipid concentration of between 10 mM and 15 mM. In some embodiments, the concentration of the first and/or second lipid concentration is greater than or equal to 10 mM, greater than or equal to 10.5 mM, greater than or equal to 11 mM, greater than or equal to 11.5 mM, greater than or equal to 12 mM, greater than or equal to 12.5 mM, greater than or equal to 13 mM, greater than or equal to 13.5 mM, greater than or equal to 14 mM, greater than or equal to 14.5 mM, or greater than or equal to 15 mM.

In some embodiments, the concentration of the first and/or second lipid composition is between about 7.5 mM and 17.5 mM, between about 8.5 mM and 16.5 mM, between about 9.5 mM and 15.5 mM, between about 10.5 mM and 14.5 mM, or between about 11.5 mM and 13.5 mM.

In some embodiments, the methods comprise mixing an API (e.g., mRNA, siRNA, dsRNA, or miRNA) with the second lipid composition. In some embodiments, the API is an siRNA.

In some embodiments, the methods comprise mixing the one or more nucleic acid with the second lipid composition prior to mixing the second lipid composition with the first lipid composition. The concentration of the nucleic acid in the second lipid composition may be any concentration that has a therapeutic effect on a subject in need thereof. In some embodiments, the concentration of the one or more nucleic acids in the second lipid composition prior to mixing the second composition with the first lipid composition is greater than or equal to 50 micrograms/mL, greater than or equal to 75 micrograms/mL, greater than or equal to 100 micrograms/mL, greater than or equal to 120 micrograms/mL, greater than or equal to 150 micrograms/mL, greater than or equal to 175 micrograms/mL, or greater than or equal to 200 micrograms/mL. In some embodiments, the concentration of the one or more nucleic acids in the second lipid composition prior to mixing the second composition with the first lipid composition is less than or equal to 200 micrograms/mL, less than or equal to 200 micrograms/mL, less than or equal to 175 micrograms/mL, less than or equal to 150 micrograms/mL, less than or equal to 120 micrograms/mL, less than or equal to 100 micrograms/mL, less than or equal to 75 micrograms/mL, or less than or equal to 50 micrograms/mL.

In some embodiments, the methods comprise mixing the one or more nucleic acid with the second lipid composition at a ratio of between 1:50 and 50:1 before mixing the second lipid composition with the first lipid composition. In some embodiments, the one or more nucleic acid is mixed with the second lipid composition at a ratio of greater than or equal to 1:1, greater than or equal to 5:1, greater than or equal to 10:1, greater than or equal to 20:1, greater than or equal to 30:1, greater than or equal to 40:1, greater than or equal to 50:1 before mixing the first lipid composition with the second lipid composition (e.g., weight percent, mole percent, etc.). In some embodiments, the ratio of the first lipid composition to the second lipid composition is less than or equal to 50:1, less than or equal to 40:1, less than or equal to 30:1, less than or equal to 20:1, less than or equal to 10:1, less than or equal to 5:1, or less than or equal to 1:1 before mixing the first lipid composition with the second lipid composition (e.g., weight percent, mole percent, etc.). Other combinations are possible in some embodiments. Other ranges are also possible in some embodiments.

In some embodiments, the methods comprise mixing the first lipid composition and second lipid composition. The first lipid composition may be added to the second lipid composition in any suitable ratio to form the LNPs disclosed herein. For example, in some embodiments, the ratio of the first lipid composition to the second lipid composition (e.g., 1^{st} lipid composition: 2^{nd} lipid composition) is greater than or equal to 1:1, greater than or equal to 0.1:1, greater than or equal to 0.2:1, greater than or equal to 0.3:1, greater than or equal to 0.4:1, greater than or equal to 0.5:1, greater than or equal to 0.5:1, greater than or equal to 0.6:1, greater than or equal to 0.7:1, greater than or equal to 0.8:1, greater than or equal to 0.9:1, greater than or equal to 1:1, greater than or equal to 1:0.9, greater than or equal to 1:0.8, greater than or equal to 1:0.7, greater than or equal to 1:0.6, greater than or equal to 1:0.5, greater than or equal to 1:0.4, greater than or equal to 1:0.3, greater than or equal to 1:0.2, greater than or equal to 1:0.1. In some embodiments, the ratio of the ratio of the first lipid composition to the second lipid composition is less than or equal to 1:0.1, less than or equal to 1:0.2, less than or equal to 1:0.3, less than or equal to 1:0.4, less than or equal to 1:0.5, less than or equal to 1:0.6, less than or equal to 1:0.7, less than or equal to 1:0.8, less than or equal to 1:0.9, less than or equal to 1:1, less than or equal to 0.9:1, less than or equal to 0.8:1, less than or equal to 0.7:1, less than or equal to 0.6:1, less than or equal to 0.5:1, less than or equal to 0.4:1, less than or equal to 0.3:1, less than or equal to 0.2:1, less than or equal to 0.1:1.

In some embodiments, the ratio of the first lipid composition to the second lipid composition is greater than or equal to 1:1, greater than or equal to 5:1, greater than or equal to 10:1, greater than or equal to 20:1, greater than or equal to 30:1, greater than or equal to 40:1, greater than or equal to 50:1. In some embodiments, the ratio of the first lipid composition to the second lipid composition is less than or equal to 50:1, less than or equal to 40:1, less than or equal to 30:1, less than or equal to 20:1, less than or equal to 10:1, less than or equal to 5:1, or less than or equal to 1:1. Other combinations are possible in some embodiments. Other ranges are also possible in some embodiments. In some embodiments, the first lipid composition and the second lipid composition comprise one or more of the same lipids (e.g., MC3, DOTAP, etc.).

In some embodiments, the methods described herein increase percent encapsulation efficiency of an API within histidine-buffered LNP compositions, relative to LNP compositions stored in phosphate buffer, after 4 weeks at 25° C. In some embodiments, the percent increase in encapsulation efficiency is between about 70% and about 140%, relative to a LNP composition stored in phosphate buffer after storage for 4 weeks at 25° C.

In some embodiments, the percent increase in the percent encapsulation efficiency of an API within histidine-buffered LNP compositions, is between 70% and 140%, between 75% and 135%, between 80% and 130%, between 85% and 125%, between 90% and 120%, between 95% and 110%, or between 100% and 105%, relative to LNP compositions stored in phosphate buffer, after 4 weeks at 25° C.

In some embodiments, the percent increase in the percent encapsulation efficiency is greater than or equal to 40%, greater than or equal to 50%, greater than or equal to 60%, greater than or equal to 70%, greater than or equal to 80%, greater than or equal to 90%, greater than or equal to 100%, greater than or equal to 120%, or greater than or equal to 140%, relative to LNP compositions stored in phosphate buffer, after 4 weeks at 25° C. . . . In some embodiments, the percent increase in the percent encapsulation efficiency is less than or equal to 140%, less than or equal to 120%, less than or equal to 100%, less than or equal to 90%, less than or equal to 80%, less than or equal to 70%, less than or equal to 60%, less than or equal to 50%, or less than or equal to 40%, relative to relative to LNP compositions stored in phosphate buffer, after 4 weeks at 25° C.

In some embodiments, the methods described herein increase the relative percent of intact ionizable lipids within a histidine-buffered LNP compositions, relative to LNP compositions stored in phosphate buffer, after 4 weeks at 25° C. Without wishing to be bound by theory, it is generally believed that mixing the negatively charged RNAs (e.g., siRNA) with the ionizable lipids in the second lipid composition (described elsewhere herein) results in the formation of RNA-lipid complexes (e.g., electrostatic bonds). These complexes act to shield the lipids from the aqueous buffer and thus decreases the incidence of water mediated hydrolysis and oxidation.

In some embodiments, the percent increase in the relative percent of intact ionizable lipids in histidine-buffered LNP compositions is between about 55% and about 75%, relative to LNP compositions stored in phosphate buffer after 4 weeks at 25° C.

In some embodiments, the percent increase in the relative percent of intact ionizable lipid in histidine-buffered LNP compositions is between about 55% and 75% or between about 60% and 70%, relative to LNP compositions stored in phosphate buffer after 4 weeks and 20° C.

In some embodiments, the percent increase in the relative percent of intact ionizable lipids in histidine-buffered LNP compositions is greater than or equal to 55%, greater than or equal to 60%, greater than or equal to 65%, greater than or equal to 70%, or greater than or equal to 75%, relative to LNP compositions stored in a phosphate buffer after 4 weeks at 25° C. In some embodiments, the percent increase in the relative percent of intact ionizable lipids in histidine-buffered LNP compositions is less than or equal to 75%, less than or equal to 70%, less than or equal to 65%, less than or equal to 60%, or less than or equal to 55%, relative to LNP compositions stored in a phosphate buffer after 4 weeks at 25° C.

In some embodiments, the methods described herein increase the relative percent of intact APIs within histidine-buffered LNP compositions, relative to LNP compositions stored in phosphate buffer, after 4 weeks at 25° C. In some embodiments, API comprises a siRNA comprising a sense strand (herein “SS-strands”) and an antisense strand (herein “AS-strands”).

In some embodiments, the percent increase in the relative percent of intact SS-strands in histidine-buffered LNP compositions is at least 100%, relative to LNP compositions stored in phosphate buffers (e.g., if the relative percent of intact SS-strands in a LNP composition comprising a phosphate buffer is ~40% after 4 weeks at 25° C., and the relative percent of intact SS-strands in a LNP composition comprising a histidine buffer is ~80%, under the same conditions, then the percent increase between the two is about ~100%.

In some embodiments, the percent increase in the relative percent of intact SS-strands in histidine-buffered LNP compositions is at least 10%, at least 20%, at least 30%, at least 40%, at least 50%, at least 60%, at least 70%, at least 80%, at least 90%, or at least 100%, relative to LNP compositions stored in a phosphate buffer after 4 weeks at 25° C.

In some embodiments, the percent increase in the relative percent of intact AS-strands in histidine-buffered LNP compositions is at least 275%, relative to LNP compositions stored in phosphate buffers (e.g., if the relative percent of intact AS-strands in a LNP composition comprising a phosphate buffer is ~20% after 4 weeks at 25° C., and the relative percent of intact SS-strands in a LNP composition comprising a histidine buffer is ~75%, under the same conditions, then the percent increase between the two is about ~275%.

In some embodiments, the percent increase in the relative percent of intact AS-strands in histidine-buffered LNP compositions is at least 10%, at least 20%, at least 30%, at least 40%, at least 50%, at least 60%, at least 70%, at least 80%, at least 90%, at least 100% at least 120%, at least 140%, at least 160%, at least 180%, at least 200%, at least 220%, at least 240%, at least 250%, or at least 275%, relative to LNP compositions stored in a phosphate buffer after 4 weeks at 25° C.

In some embodiments, the methods described herein preserve the biological function of APIs encapsulated within histidine-buffered LNP compositions, relative to LNP compositions stored in phosphate buffers, after 4 weeks of storage at 25° C. In some embodiments, the methods described herein increase the inhibitory concentration (e.g., IC50) of siRNAs encapsulated within histidine-buffered LNP compositions, relative to LNP compositions stored in phosphate buffers, after 4 weeks of storage at 25° C. In some embodiments, siRNAs encapsulated within histidine-buffered LNP compositions exhibit an inhibitory concentration of at least 5 picoM, at least 10 picoM, at least 20 picoM, at least 30 picoM, at least 40 picoM, or at least 50 picoM after storage for 4 weeks at 25° C. In some embodiments, siRNAs encapsulated within histidine-buffered LNP compositions exhibit an inhibitory concentration of at least 50 picoM. In some embodiments, siRNAs encapsulated within LNP compositions stored in phosphate buffers lack an inhibitory concentration after storage for 4 week at 25° C. (e.g., they are completely degraded an exhibit no biological activity).

Aspects of the disclosure relate to compositions and methods useful for treating diseases or disorders associated with dysregulated expression of mRNA and/or the protein products they encode. In some embodiments, the compositions and methods described herein modulate the function, activity, and/or level the protein product encoded by the target mRNA by decreasing target mRNA level and/or translation of that target mRNA in a cell or subject.

### EXAMPLES

### Background

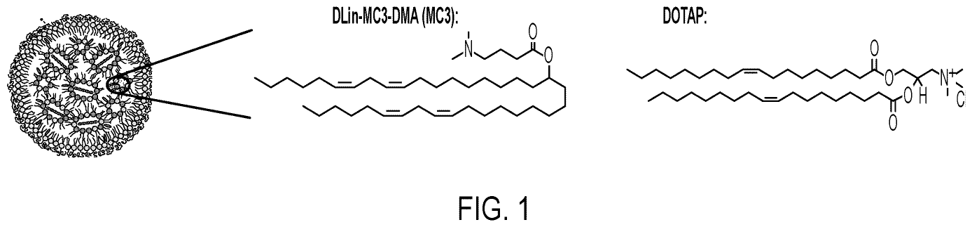

The stability of ionizable lipids, MC3 and DOTAP (FIG. 1), to hydrolytic and oxidative degradation when stored in phosphate buffers was investigated.

Stock MC3 lipidic solutions were prepared by dissolving MC3 in an ethanolic solution to a final concentration of 4 mg/mL (e.g., 6 mM). Similarly, stock lipidic DOTAP solutions were prepared by dissolving DOTAP in an ethanolic solution to a final concentration of 4 mg/mL. Stock solutions of phosphate buffered saline (1×, pH 7.4) were used. Final lipidic solutions were prepared by mixing PBS stock to the lipidic stock in a ratio of 3:1 (e.g., PBS stock:DOTAP stock in ethanol=3:1; PBS stock:MC3 stock in ethanol=3:1). Solutions were stored in either glass (BT5933) or polymer (COP) vials.

Dilute lipidic solutions were frozen at −70° C. before being thawed and stored at either 5° C., 25° C., or 40° C. for either 1 week, 2 weeks, or 4 weeks. At each timepoint, (e.g., after 1 week, 2 weeks, or 4 weeks) the lipid solution was analyzed via LC-MS to determine the extent of hydrolysis and/or oxidation present.

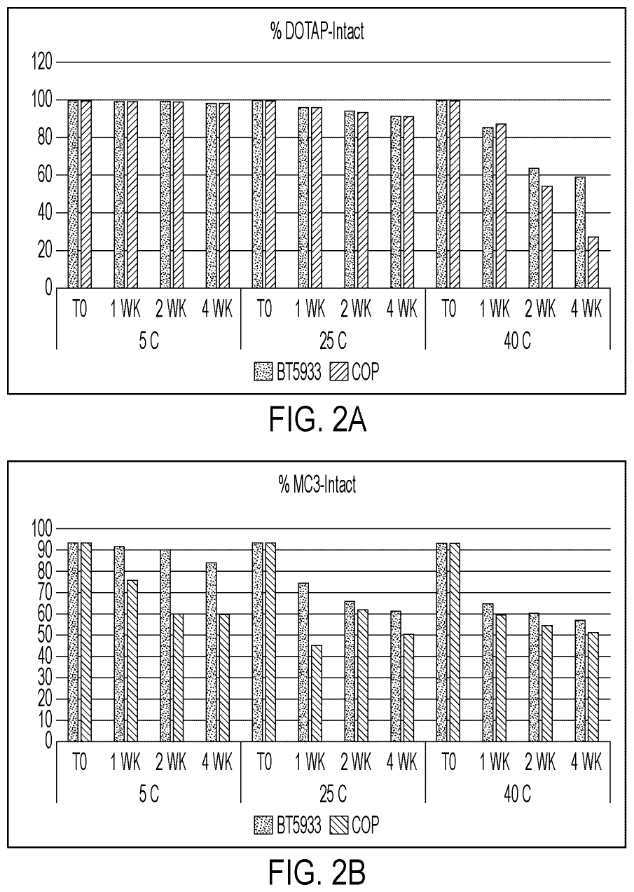

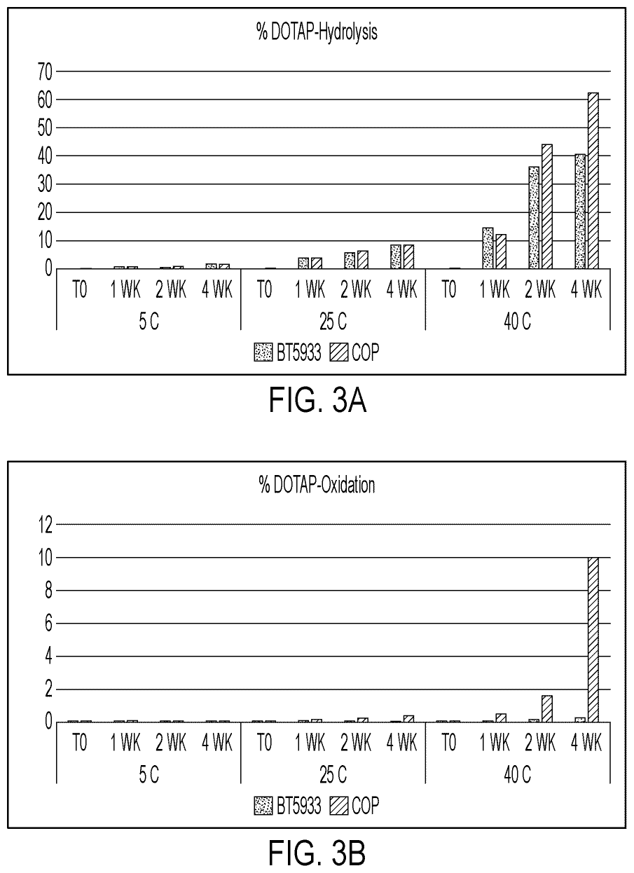

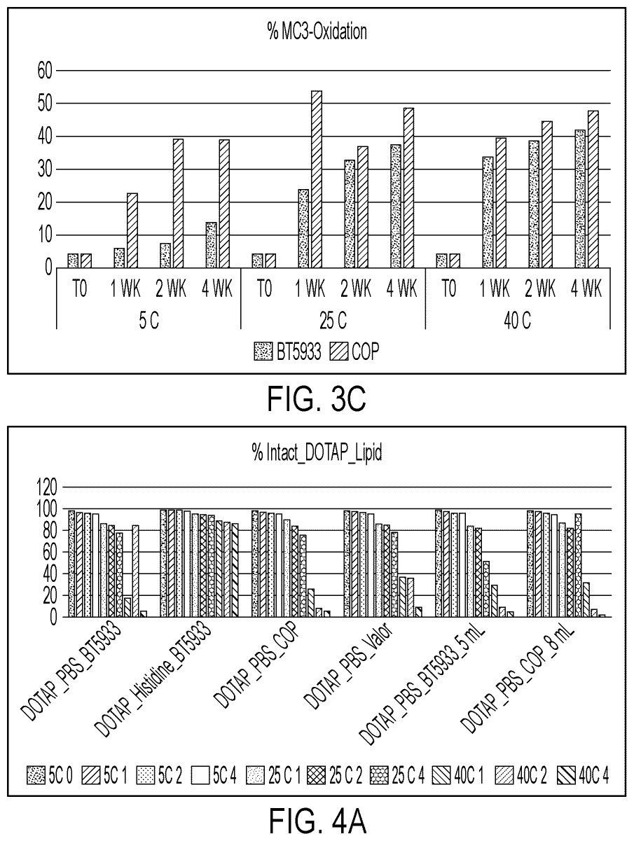

As shown in FIG. 2A, the % DOTAP that remained intact (e.g., was not degraded) decreased with increasing storage time (e.g., 1 week >4 week) and was lowest for samples stored at 40° C. Also, samples stored in BT5933 glass vials exhibited better stability compared to identical samples stored in COP vials when diluted with PBS buffer. LC-MS analysis indicated that the primary degradation pathway for DOTAP is hydrolysis (FIG. 3A) with oxidation only contributing to degradation at prolonged storage times and elevated storage temperature (FIG. 3B, see storage at 40° C. for 4 weeks).

FIG. 2B, shows the percentage (%) MC3 intact for the various test groups. The % MC3 remaining intact following storage at 5° C. and 25° C., was lower than the % DOTAP for the same time points, suggesting that MC3 degrades faster than DOTAP under these storage conditions. As above, storage in the BT5933 glass vials improved stability relative to COP vials when diluted in PBS buffer. LC-MS analysis indicated that the primary degradation pathway for MC3 is oxidation at all conditions tested (see FIG. 3C). Data indicate hydrolysis is the major degradation mechanism for DOTAP (FIG. 3A).

### Example 1. Lipid Chemical Stability in Histidine Buffers Study

MC3 and DOTAP lipids were prepared using either PBS or Histidine buffers. Ethanolic stock solutions of MC3 and DOTAP were prepared as described in Example 1. PBS-based solutions were prepared by diluting the stock lipid solutions, respectively, by 3:1 (PBS:lipid in ethanol (vol/vol)) using 1× PBS, pH 7.4. Histidine-based solutions were also prepared by diluting the stock lipidic solution by 3:1 (histidine:lipid in ethanol vol/vol) using a histidine buffer (10 mM, pH 6.0). Solutions were filled into BT5933, COP, or Valor glass vials at 1 mL aliquots and put on stability. Initial control samples were immediately frozen at −70° C.

Filled vials were stored at either 5° C., 25° C., or 40° C. for either 1 week, 2 weeks, or 4 weeks. At each timepoint, (e.g., after 1 week, 2 weeks, or 4 weeks) the lipid solution was analyzed via LC-MS to determine the extent of hydrolysis and/or oxidation present.

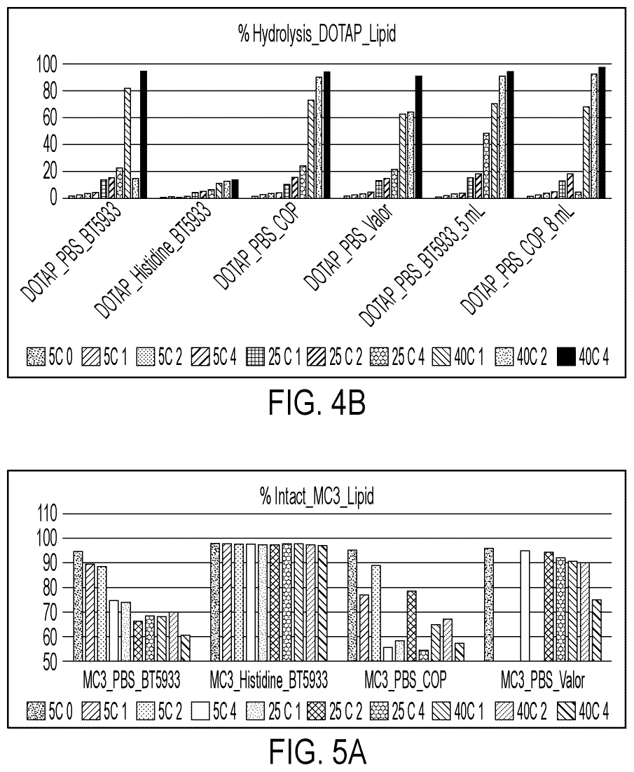

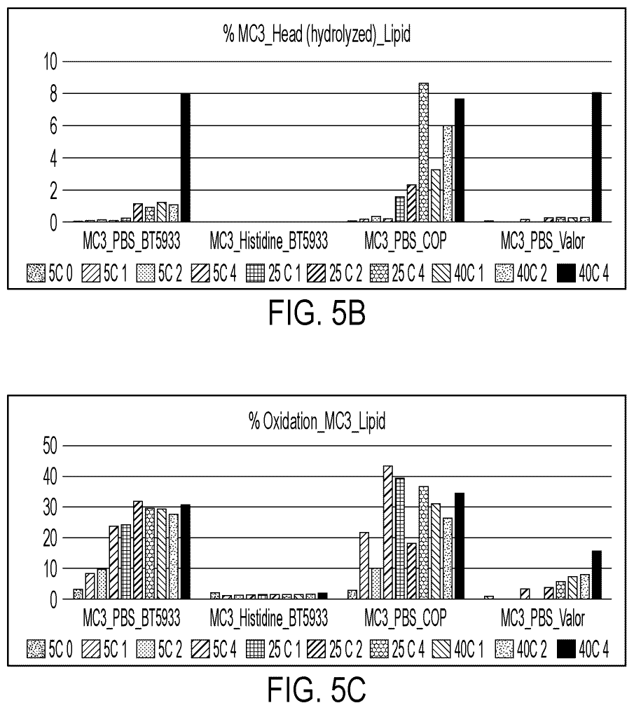

As shown in FIG. 4A, storage in histidine buffers stabilized the % DOTAP intact at all storage conditions tested, and also, decreased the % hydrolysis of DOTAP relative to storage in PBS, regardless of the storage conditions (FIG. 4B). Additionally, storage in histidine buffers significantly reduced lipid hydrolysis of MC3 (FIG. 5B) and reduced the degree of oxidation (FIG. 5C), thus increasing the % MC3 intact at all storage conditions tested (FIG. 5A). The data further indicate that storage in Valor glass vials results in the lower oxidation rate for MC3.

### Example 2. Empty LNP Chemical and Colloidal Stability Study

The stability of empty LNP comprising ionizable lipids, MC3 and DOTAP, to hydrolytic and oxidative degradation when stored in either phosphate buffers or histidine buffers was investigated.

Empty MC3-LNPs and DOTAP-LNPs were prepared in either PBS (1×, pH 7.4) or Histidine buffer (20 mM histidine, 140 mM NaCl, pH 6.0) and stored in either a BT5933 (5 mL, 51 COE, Schott) glass vial with a VS5558 cap, a BT5974 (5 mL, 33 COE, Gerresheimer treated) glass vial with a VS5558 cap, a COP vial (5 mL) with matching stopper, Valor vials with matching cap, or BT5933 Schott vials with VS5558 caps.

LNP solutions were stored at either 5° C. or 25° C., for either 0 days, 1 week, 2 weeks, 3 weeks, or 4 weeks, and then frozen at −70° C. At each timepoint, the LNP solution was analyzed via LC-MS to determine the LNP size (via dynamic light scattering), and degradation via hydrolysis and/or oxidation present (via LC-MS)

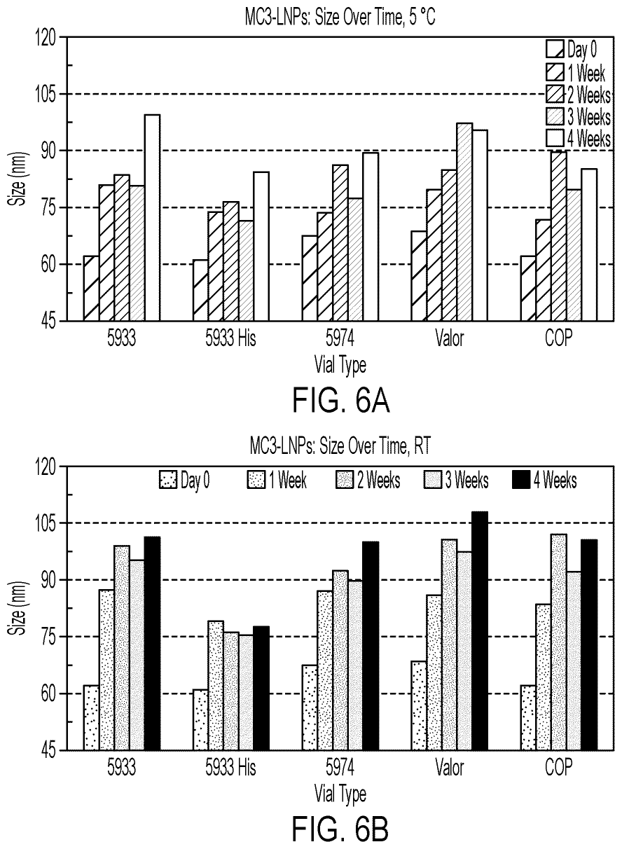

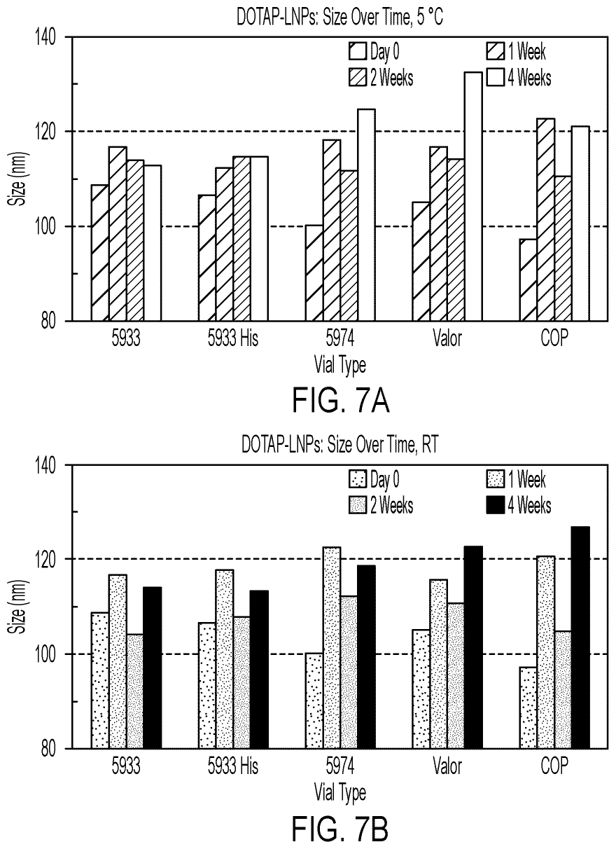

As shown in FIG. 6A, the composition of the storage buffer had little effect on MC3-LNP size when stored at 5° C. In contrast, FIG. 6B shows that histidine buffers improved colloidal stability of MC3-LNPs at RT for up to 4 weeks, with the maximum LNP size reaching approximately 80 nm, compared to 100-110 nm for identical LNPs stored in PBS-based buffers. FIGS. 7A and 7B show that the mean particle size of DOTAP-LNPs is larger than MC3-LNPs regardless of the storage conditions.

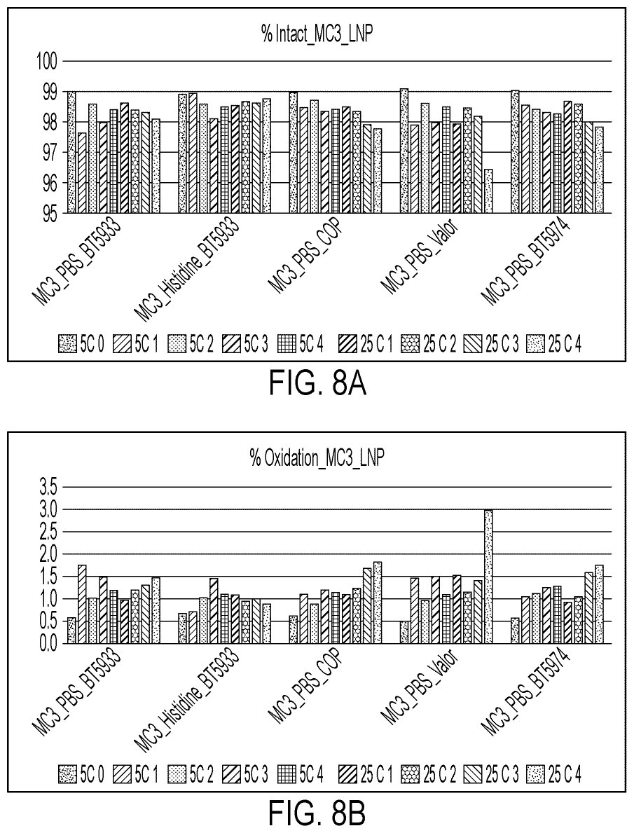

FIGS. 8A and 8B show that the improved size control of MC3-LNPs stored in histidine buffers (FIG. 6B) was associated with a slight increase in the percentage of intact MC3-LNPs following the 4-week storage period, the result of which, was due to a slight reduction in the percent oxidation of MC3 in the MC3-LNPs (FIG. 8B).

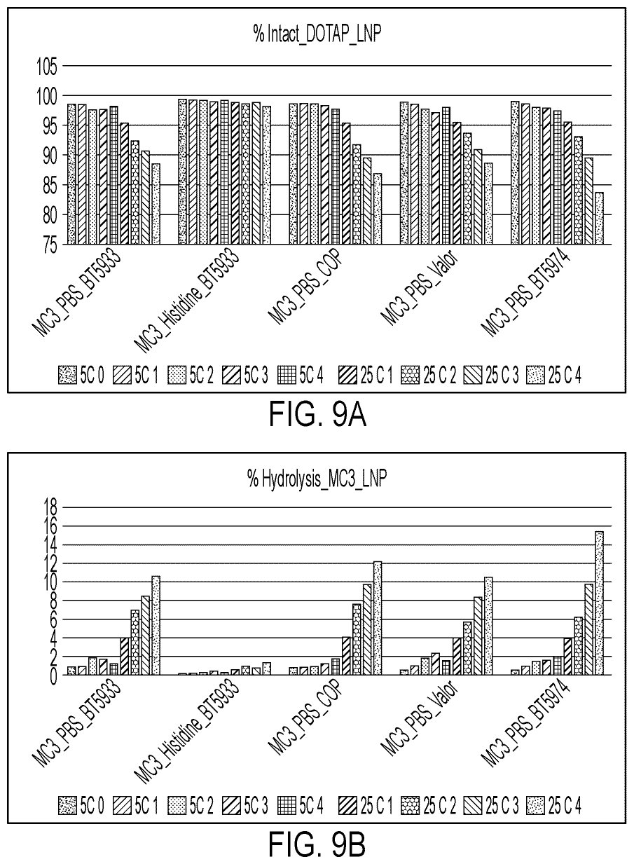

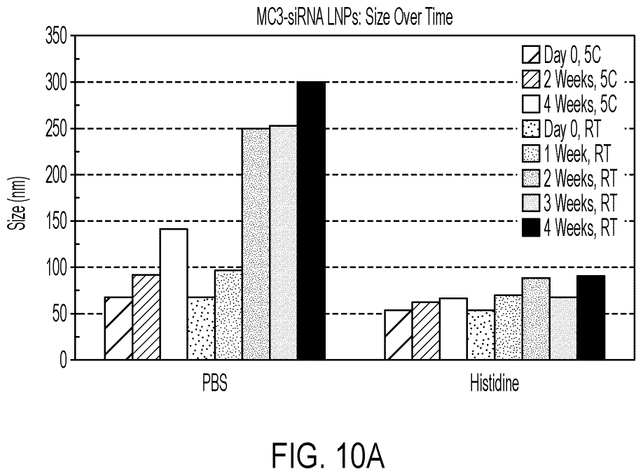

FIGS. 9A and 9B show storage in histidine buffer increased the percentage of intact DOTAP-LNPs following the 4-week storage period at RT (FIG. 9A), the result of which, was due to a reduction in the percent hydrolysis of DOTAP in the DOTAP-LNPs (FIG. 9B).

### Example 3. SiRNA-Loaded LNP Chemical and Colloidal Stability Study

The stability of siRNA-loaded LNPs comprising ionizable lipids, MC3 and DOTAP, to hydrolytic and oxidative degradation when stored in either phosphate buffers or histidine buffers was investigated.

LNPs were prepared using a composition containing Dlin-MC3-DMA, Cholesterol, DSPC, DMG-PEG-2k in a ratio of 50:38.5:10:1.5 on a mole % basis. The composition was mixed with MC3 or DOTAP lipids at a concentration of 12.5 mM, 50 mM sodium citrate (pH 5), and siRNA (siHPRT) at a concentration of 0.12 ng/ml (20:1 wt %). This solution was then passed through a ISCO pump (Teledyne Inc.) and a PD-10 column to yield the desired LNPs. LNPs were then diluted at a volume of 1:1 with either PBS (1×, 137 mM NaCl, 2.7 mM KCl, 10 mM Na₂HPO₄, and 1.8 mM KH₂PO₄ pH 7.4) or Histidine buffer (20 mM histidine, 140 mM NaCl, pH 6.0) to produce the final LNP solution. Both samples were stored in 5933 Schott, 5 mL, 51 COE glass vials.

LNP solutions stored at either 5° C. for either 0 days, 2 weeks or 4 weeks; or at RT for 0 days, 1 week, 2 weeks, 3 weeks, 4 weeks, or 3 months, and then frozen at −70° C. At each timepoint, the LNP solution was analyzed via dynamic light scattering (e.g., size determination), polydispersity index (e.g., PDI), encapsulation efficiency, and LC-MS (e.g., stability) to determine the extent of hydrolysis and/or oxidation present.

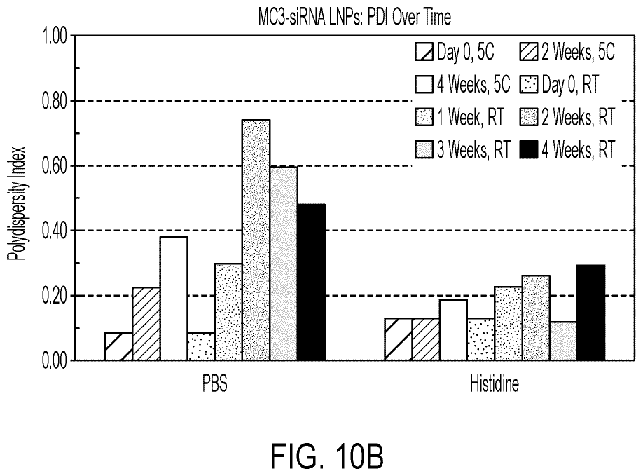

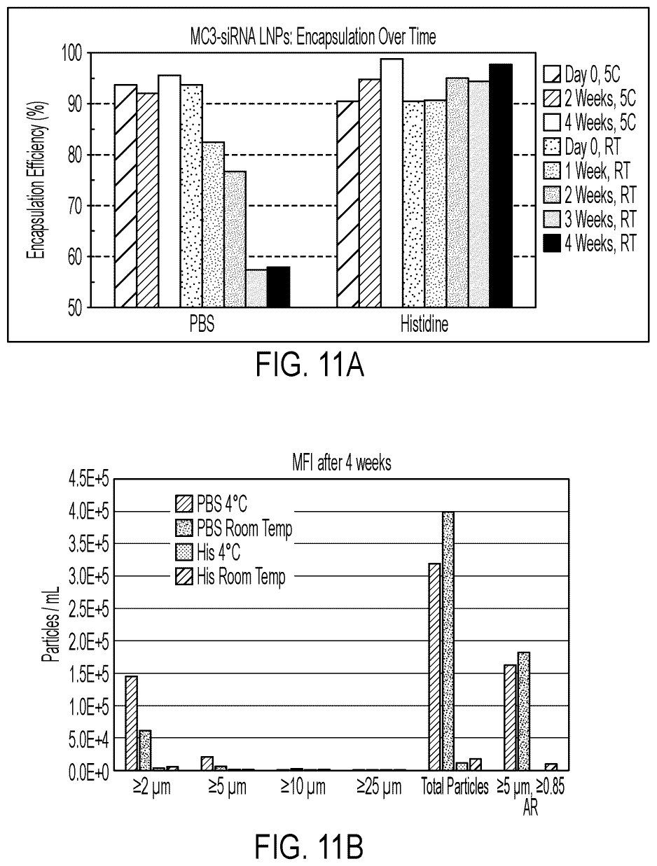

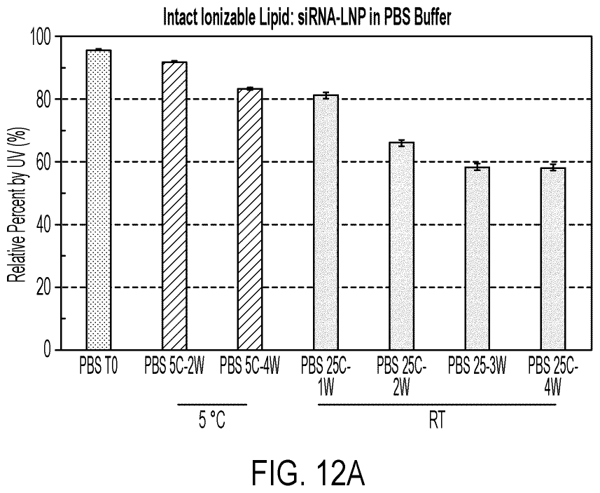

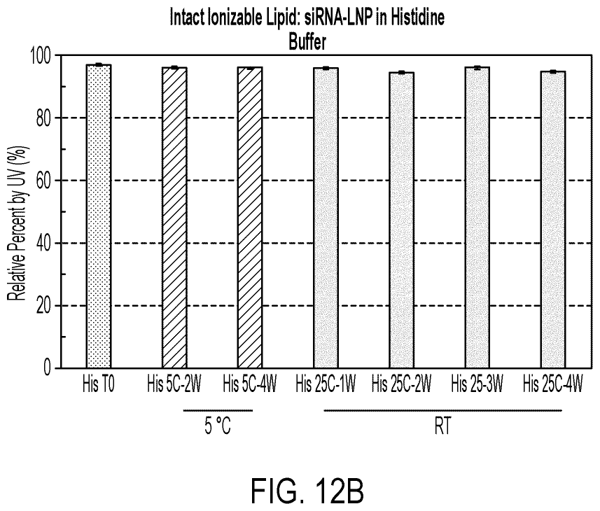

FIGS. 10A and 10B show that storage in histidine buffers produced MC3-LNPs with smaller particle sizes (FIG. 10A) and helped maintain low and relatively constant PDIs (FIG. 10B). FIGS. 11A and 11B show that storage in the histidine buffer increased the encapsulation efficiency at both 5° C. and RT starting at 2 weeks and continuing until the 4-week period; storage in the histidine buffer also resulted in much less subvisible particles with diameters larger than or equal to 2 microns (FIG. 11B). Encapsulation efficiency was determined by Quant-it™ RiboGreen RNA Assay Kit. FIGS. 12A and 12B show that storage in the histidine buffer increased the percentage of intact MC3-LNPs (FIG. 12B) to over 95% after the 4-week observation period, regardless of temperature, in contrast to those stored in PBS (~58%, FIG. 12A).

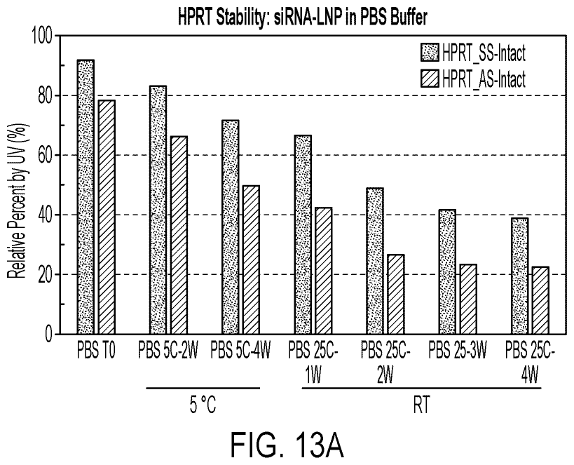

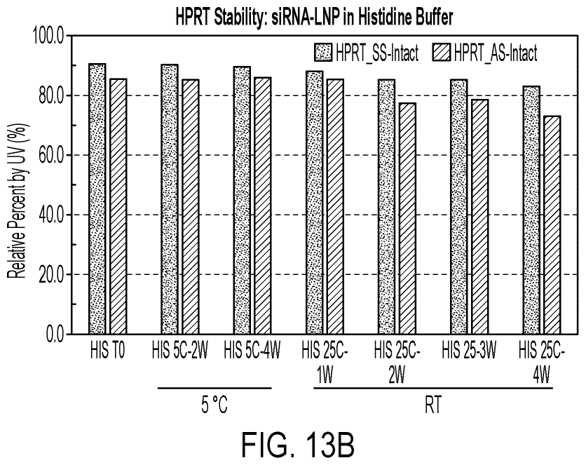

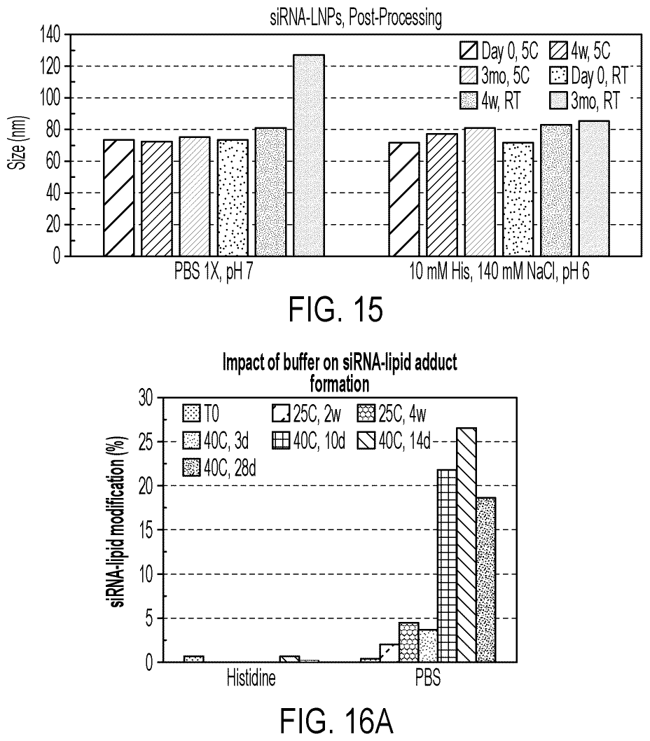

Unexpectedly, storage of siRNA-loaded LNPs in histidine buffers also increased the stability of the encapsulated siRNAs, relative to those stored in PBS under similar conditions. For example, FIGS. 13A and 13B show that double stranded siHPRT degrades over the 4-week storage period when encapsulated within siRNA-LNPs formulated in PBS buffers (FIG. 13A); compared to siHPRT encapsulated in LNPs formulated using histidine buffer (FIG. 13B). After 3 months of storage at RT, histidine buffered siRNA-LNPs remained significantly more stable than phosphate buffered compositions (FIG. 15). Properties of siRNA-loaded LNPs was also assessed after storage at 40° C. Data indicate that histidine-buffered compositions were stable even after 4 weeks of storage at elevated temperatures (Table 1).

TABLE 1

| | 40° C. | | 25° C. | |
|---|---|---|---|---|
| | 10 mM His | 1X PBS | 10 mM His | 1X PBS |
| | pH 6.0 | pH 7.4 | pH 6.0 | pH 7.4 |
| Intact MC3 | 99 | 74 | 100 | 97 |
| Lipid (≥90%) | | | | |
| Intact Sense | 93 | 26 | 96 | 87 |
| Strand (≥80%) | | | | |
| Intact | 85 | 12 | 93 | 66 |
| Antisense | | | | |
| Strand (≥80%) | | | | |
| Size (≤100 nm) | 81 | 112 | 75 | 81 |
| Polydispersity | 0.10 | 0.40 | 0.18 | 0.23 |
| Index (≤0.30) | | | | |
| Encapsulation | 99 | 86 | 97 | 74 |
| Efficiency | | | | |
| (≥90%) | | | | |
| Transmittance | 28 | 28 | 31 | 19 |
| (≥20%) | | | | |

### Example 4. In Vitro Knock-Down Efficiency Study

The ability of siRNA-loaded LNPs to knock down a gene of interest in vitro following storage under a variety of different conditions was next evaluated.

MC3-LNPs encapsulating siHPRT were prepared as described in Example 3 and stored under one of the conditions shown in Table 1 until needed. To test the effect of storage conditions on the transfection efficiency, HeLa cells were added to a 96-well plate (10,000 cells/well) and the appropriate LNP solution (e.g., condition and concentration), or negative control, was added to each well. Each group in Table 2 was tested at 6 different concentrations of RNA within the LNPs (e.g., 100 mM, 10 mM, 1 mM, 0.1 mM, 0.01 mM, and 0 mM RNA within the LNP). Thus, each group required 6 wells per repeat and each experiment was repeated 3× (e.g., each Group required 18 wells). Plates were allowed to incubate for 24 hours at 37° C. and 95/5 O₂/CO₂. Transfection efficiency was subsequently determined by isolating cDNA from each well and the concentration of HPRT mRNA determined via qPCR.

TABLE 2
Conditions for in cellulo study

| Group | API | Buffer | Temperature | Time | | Concentration |
|---|---|---|---|---|---|---|
| 1 | MC3-LNP | PBS | RT | 0 | days | 100 mM, 10 mM, |
| 2 | MC3-LNP | PBS | RT | 4 | wks | 1 mM, 0.1 mM, |
| 3 | MC3-LNP | His | RT | 4 | wks | 0.01 mM, and 0 mM |
| 4 | MC3-LNP | PBS | 4° C. | 4 | wks | |
| 5 | MC3-LNP | His | 4° C. | 4 | wks | |

| | | | | |
|---|---|---|---|---|
| 6 | siHPRT only | — | — | — |
| 7 | RNAiMax | — | — | — |
| | transfected | | | |
| | siHPRT | | | |

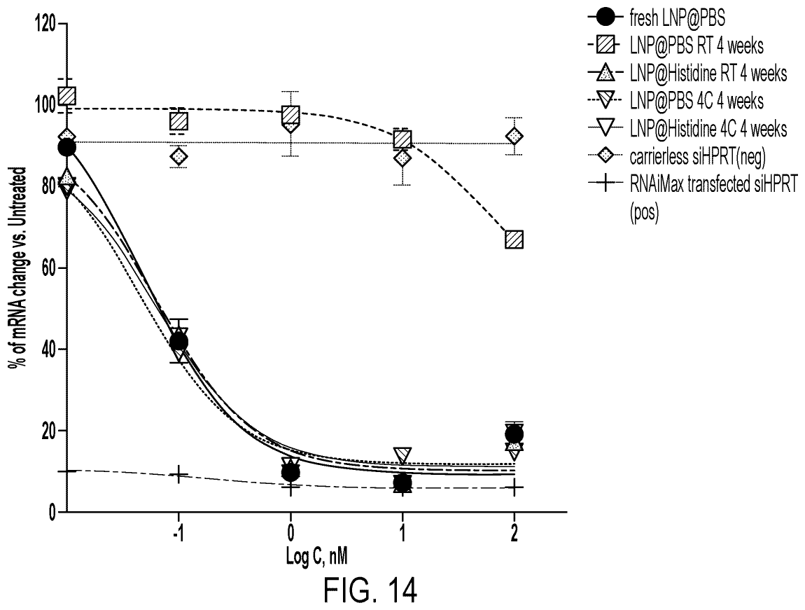

FIG. 14 shows that LNPs stored in histidine buffer at RT for 4 weeks had similar IC50 values as freshly prepared LNPs (Group 1) and LNPs formulated in PBS and stored at 4° C. for 4 weeks. Thus, LNP solutions formulated in histidine buffer and stored at either 4° C. or RT maintained the biological function of siHPRT, relative to LNPs formulated in PBS and stored at RT.

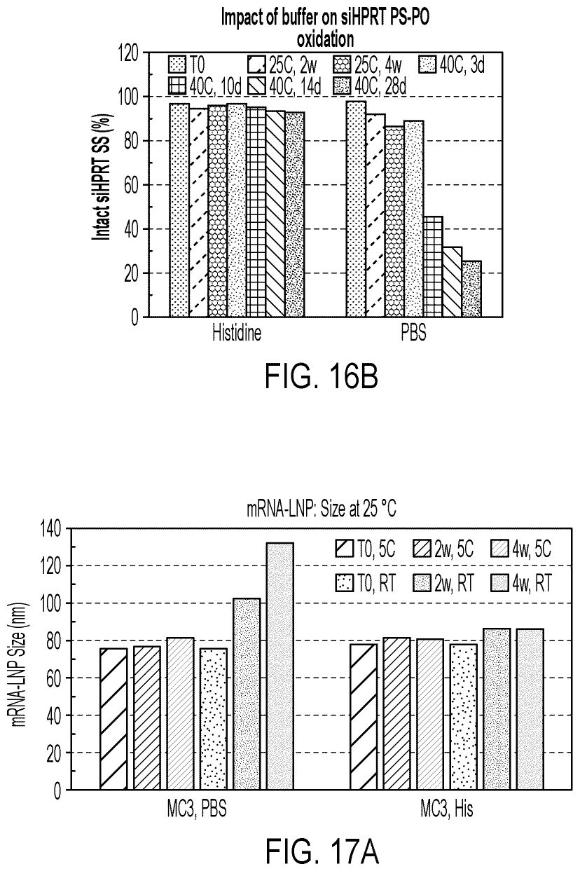

FIG. 16A shows that storage in histidine buffer results in formation of fewer siRNA-lipid adducts during storage at a variety of temperatures relative to phosphate buffered compositions. FIG. 16B shows representative data indicating that histidine buffer storage inhibits oxidation of phosphorothioate linkages (PS) to phosphodiester (PO) linkages in chemically-modified siRNAs, resulting in fewer siRNA-lipid adducts.

### Example 5. MRNA-Loaded LNPs

This example describes the effects of histidine buffering on mRNA-loaded LNP colloidal and payload stability. Briefly, mRNAs were formulated using 8 mM Dlin-MC3-DMA at an N/P ratio of 6, and stored in either phosphate buffer (PBS), pH 7.4, or histidine buffer pH 6.0 for 2 weeks or 4 weeks, either at room temperature or 5° C. Compositions were then characterized by MFI, turbidity (via UV), osmolarity, DLS, RiboGreen assay, and CryoEM.

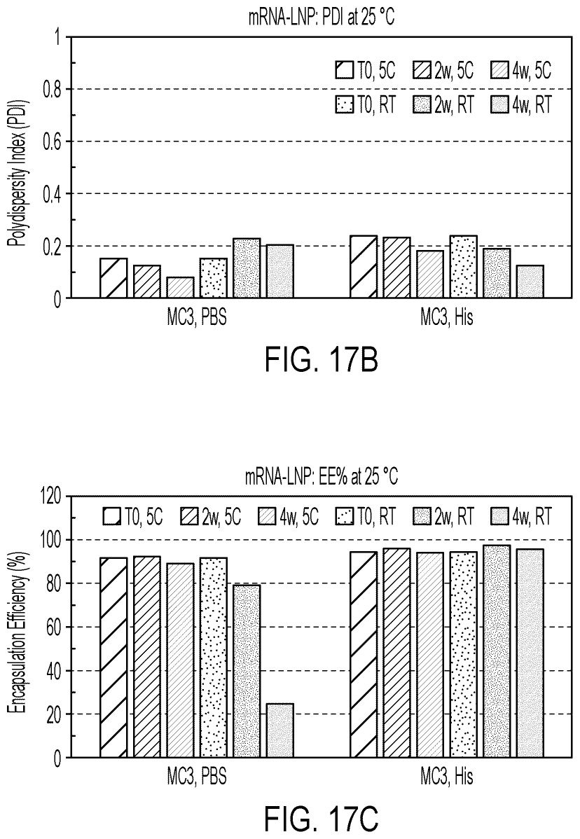

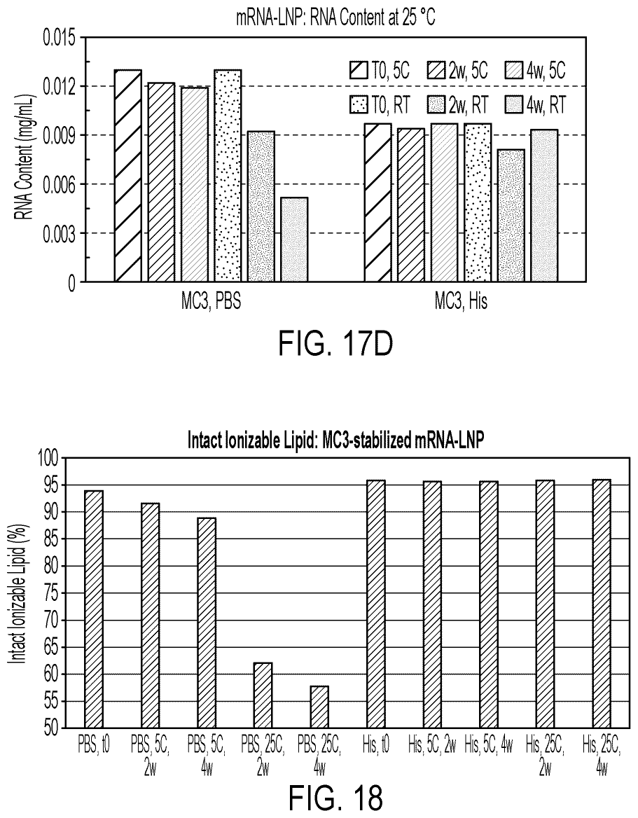

FIGS. 17A-17D show representative data for measurement of mRNA-LNP colloidal and payload stability. FIG. 17A shows mRNA-LNP size at 25° C. FIG. 17B shows mRNA-LNP polydispersity index (PDI) at 25° C. FIG. 17C shows mRNA-LNP encapsulation efficiency (EE) at 25° C. FIG. 17D shows mRNA-LNP RNA content at 25° C. RNA content was observed to be reduced in PBS stored compositions.

FIG. 18 shows representative data indicating that MC3-stabilized mRNA-LNPs experience a similar level of ionizable lipid degradation as siRNA-LNPs, and that storage in histidine buffer prevents lipid degradation.

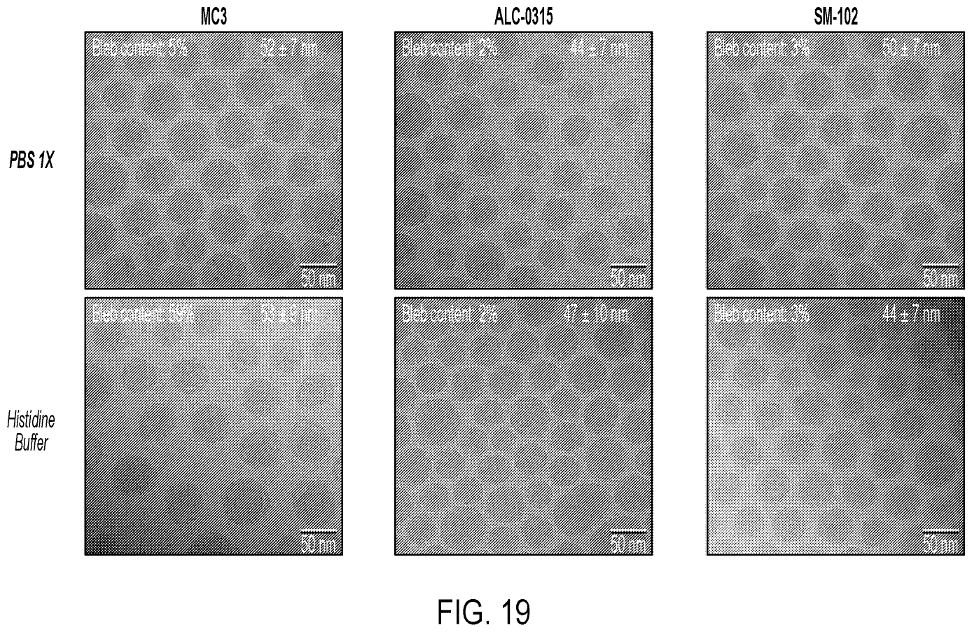

FIG. 19 shows representative cryogenic electron microscopy (CryoEM) images.

What is claimed is:

1. A pharmaceutical composition comprising:
(i) a lipid nanoparticle (LNP) comprising one or more ionizable lipids; and
(ii) a histidine buffer having a concentration ranging from 5 mM to 30 mM histidine and a pH ranging from about 5.0 to about 7.5.

2. The pharmaceutical composition of claim 1, wherein the concentration of histidine buffer ranges between 10 mM and 20 mM histidine.

3. The pharmaceutical composition of claim 1 or 2, wherein the concentration of histidine buffer is at least 10 mM.

4. The pharmaceutical composition of any one of claims 1 to 3, wherein the concentration of histidine buffer is 20 mM.

5. The pharmaceutical composition of any one of claims 1 to 4, wherein the pH of the histidine buffer is about pH 6.0.

6. The pharmaceutical composition of any one of claims 1 to 5, wherein the histidine buffer further comprises one or more salts.

7. The pharmaceutical composition of claim 6, wherein the one or more salts comprises NaCl.

8. The pharmaceutical composition of any one of claims 1 to 7, wherein the one or more ionizable lipids comprises an unsaturated tail, further optionally wherein the one or more ionizable lipids comprises DLin-MC3-DMA (MC3) and/or Dioleoyl-3-trimethylammonium propane (DOTAP).

9. The pharmaceutical composition of claim 8, wherein the one or more ionizable lipid consists of MC3 or DOTAP.

10. The pharmaceutical composition of any one of claims 1 to 9, wherein the LNP comprises one or more nucleic acids.

11. The pharmaceutical composition of claim 10, wherein the one or more nucleic acids comprises RNA.

12. The pharmaceutical composition of claim 11, wherein the RNA is mRNA, siRNA, dsRNA, or miRNA.

13. The pharmaceutical composition of claim 11 or 12, wherein the RNA is siRNA.

14. The pharmaceutical composition of any one of claims 1 to 13, wherein the composition has not been refrigerated or frozen.

15. The pharmaceutical composition of any one of claims 1 to 14, wherein the composition is stored at a temperature above 4° C.

16. The pharmaceutical composition of claim 15, wherein the temperature above 4° C. ranges from about 5° C. to about 30° C.

17. A container containing the pharmaceutical composition of any one of claims 1 to 16.

18. The container of claim 17, wherein the container is a prefilled syringe or a glass vial.

19. A method for improving chemical stability of a lipid nanoparticle (LNP) pharmaceutical composition, the method comprising:
(i) obtaining a non-histidine-buffered LNP pharmaceutical composition comprising a non-histidine buffer; and
(ii) performing a buffer exchange procedure to replace the non-histidine buffer with a histidine buffer having a pH between 5.8 and 7.5 to obtain a histidine-buffered LNP pharmaceutical composition.

20. The method of claim 19, wherein the histidine-buffered LNP pharmaceutical composition comprises one or more ionizable lipids comprises DLin-MC3-DMA (MC3) and/or Dioleoyl-3-trimethylammonium propane (DOTAP).

21. The method of claim 19 or 20, wherein the non-histidine buffer comprises a citrate buffer.

22. The method of claim 21, wherein the citrate buffer has a pH ranging from about 3.5 to about 5.5.

23. The method of claim 22, wherein the citrate buffer has a pH of 5.0.

24. The method of any one of claims 19 to 23, wherein the histidine-buffered LNP pharmaceutical composition comprises one or more nucleic acids.

25. The method of claim 24, wherein the one or more nucleic acids comprises RNA.

26. The method of claim 25, wherein the RNA is mRNA, siRNA, dsRNA, or miRNA.

27. The method of claim 25 or 26, wherein the RNA is siRNA.

28. The method of any one of claims 19 to 27, wherein the histidine buffer has a pH of 6.0.

29. The method of any one of claims 19 to 28, wherein the histidine buffer has a concentration of histidine ranging from about 5 mM to about 30 mM.

30. The method of any one of claims 19 to 29, wherein the histidine buffer has a concentration of histidine ranging from about 10 mM and 20 mM.

31. The method of claim 29 or 30, wherein the concentration of histidine is at least 10 mM.

32. The method of claim 29 or 30, wherein the concentration of histidine is 20 mM.

33. The method of any one of claims 19 to 32, wherein the buffer exchange procedure comprises contacting the non-histidine-buffered LNP pharmaceutical composition to a de-salting column.

34. The method of any one of claims 19 to 32, wherein the buffer exchange procedure comprises contacting the non-histidine-buffered LNP pharmaceutical composition to a dialysis tube or performing tangential flow filtration.

35. The method of any one of claims 19 to 34, wherein the buffer exchange procedure comprises collecting the histidine-buffered LNP pharmaceutical composition in a container.

36. The method of claim 35, wherein the container is a syringe or a glass vial.

37. The method of any one of claims 19 to 36, further comprising storing the histidine-buffered LNP pharmaceutical composition at a temperature above 4° C.

38. The method of claim 37, wherein the temperature above 4° C. ranges from about 5° C. to about 30° C.

39. The method of any one of claims 19 to 38, wherein the histidine-buffered LNP pharmaceutical composition comprises fewer hydrolyzed lipids relative to a non-histidine-buffered LNP pharmaceutical composition stored in a phosphate buffer.

40. The method of any one of claims 19 to 39, wherein the histidine-buffered LNP pharmaceutical composition comprises fewer oxidized lipids relative to a non-histidine-buffered LNP pharmaceutical composition stored in a phosphate buffer.

41. The method of any one of claims 19 to 40, wherein the histidine-buffered LNP pharmaceutical composition comprises LNPs having increased colloidal stability relative to non-histidine-buffered LNPs stored in a pharmaceutical composition comprising phosphate buffer.

42. A method for preparing a lipid nanoparticle (LNP) pharmaceutical composition, the method comprising:
(i) preparing a first lipid composition comprising one or more lipids;
(ii) preparing a second lipid composition comprising a siRNA and an ionizable lipid, and
(iii) mixing the first lipid composition and the second lipid composition, wherein the first lipid composition and second lipid composition are prepared using a histidine buffer having a pH between 5.8 and 6.5 and a histidine concentration of between 5 mM to 30 mM.

43. A method for preparing a lipid nanoparticle (LNP) pharmaceutical composition, the method comprising:
(i) obtaining a lipid composition comprising a siRNA and an ionizable lipid, and;
(ii) mixing the lipid composition with a histidine buffer having a pH between 5.8 and 6.5 and a histidine concentration of between 5 mM to 30 mM.
